(19) United States
(12) Patent Application Publication
(10) Pub. No.: US 2002/0132350 A1
(43) Pub. Date: Sep. 19, 2002

(54) Targeted genetic manipulation using Mu bacteriophage cleaved donor complex

(75) Inventors: Hideki Suzuki, Fremont, CA (US); Leszek Alexander Lyznik, Johnston, IA (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Pioneer Hi-Bred International, Inc.

(21) Appl. No.: 09/951,856

(22) Filed: Sep. 13, 2001

Related U.S. Application Data
(60) Provisional application No. 60/233,057, filed on Sep. 14, 2000.

(51) Int. Cl. C12N 15/74; C12N 15/00

(52) U.S. Cl. 435/476; 435/320.1

(57) ABSTRACT

Compositions and methods for targeted genetic manipulation of an organism are provided. The compositions are novel integration vectors derived from the Mu bacteriophage comprising an active cleaved donor complex (CDC) and further comprising a targeting mechanism whereby integration of the Mu transposable cassette may be directed to a predetermined target site within a host organism's genome. These integration vectors comprise a Mu transposable cassette and one or more navigator elements that direct targeted insertion of the CDC. Methods of the invention utilize the integration vectors of the invention to insert the Mu transposable cassette into a target site of an organism's genome. This insertion occurs in the absence of the MuB accessory protein. The methods are useful for modulating activity of known genes and for targeting integration of nucleotide sequences of interest into a specific location of an organism's genome. Accordingly, the methods may also be used to create gene disruptions and knockouts.

### FIELD OF THE INVENTION

The invention relates to the field of genetic engineering, specifically to targeted manipulation of gene expression in organisms.

### BACKGROUND OF THE INVENTION

Genetic modification techniques enable one to insert exogenous nucleotide sequences into an organism's genome to alter the phenotype of the organism. Depending upon the desired outcome of the modification, manipulation of a genome may be aimed at creating, enhancing, decreasing, or even disrupting the production of a functional gene product.

A number of methods have been described and utilized to produce stably transformed prokaryotic and eukaryotic cells. All of these methods are based on introducing a foreign DNA into a host cell and subsequent isolation of those host cells containing the foreign DNA integrated into the genome. Unfortunately, such methods produce transformed cells that contain the introduced foreign DNA inserted randomly throughout the genome, often in multiple copies. Similarly, various methods have been employed to inactivate a gene product, including efforts at gene disruption and partial or complete gene deletions, but the success of such techniques in vivo has been hampered by the non-homologous recombination that typically results. The random insertion of introduced DNA into the genome of host cells can be lethal if the foreign DNA happens to insert into, and thus mutate, a critically important native gene. In addition, even if a random insertion event does not impair the functioning of a host cell gene, the expression of an inserted foreign gene may be influenced by “position effects” caused by the surrounding genomic DNA. In some cases, a foreign gene may be inserted into sites where the position effects are strong enough to prevent the synthesis of an effective amount of product from the introduced gene. In other instances, overproduction of the foreign gene product has deleterious effects on the cell.

Homologous recombination has been used to to regulate gene expression and to generate knockout mutants in prokaryotic systems. However, foreign nucleotide sequences transferred into eukaryotic host cells undergo homologous recombination with homologous endogenous host sequences only at very low frequencies and are so inefficiently recombined that large numbers of cells must be transfected, selected, and screened in order to generate a desired correctly targeted homologous recombinant (Kucherlapati et al. (1984) Proc. Natl. Acad. Sci. USA 81: 3153; Smithies (1985) Nature 317: 230; Song et al. (1987) Proc. Natl. Acad. Sci. USA 84: 6820; Doetschman et al. (1987) Nature 330: 576; Kim and Smithies (1988) Nucleic Acids Res. 16: 8887; Shesely et al. (1991) Proc. Natl. Acad. Sci. USA 88: 4294; Kim et al. (1991) Gene 103: 227). This is particularly true in plants, where past attempts have relied on the more poorly characterized recombination activities of the plant cell system to introduce a piece of foreign DNA into the plant genome. Efficiency of DNA transfer using the native plant cellular recombination mechanism remains a limiting factor for stable integration of foreign DNA into a plant genome and generation of a sufficient number of transformation events.

For higher eukaryotes, homologous recombination is an essential event participating in processes like DNA repair and chromatid exchange during mitosis and meiosis. Recombination depends on two highly homologous extended sequences and a number of auxiliary proteins. Strand separation can occur at any point between the regions of homology, although particular sequences may influence efficiency. These processes have been exploited for targeted integration of transgenes into the genome of certain cell types. An essential feature of homologous recombination is that the auxiliary proteins responsible for the actual recombination event presumably use any pair of homologous sequences as substrates, with some types of sequences being favored over others.

Site-specific recombination offers an alternative method for insertion of a foreign nucleotide sequence into chromosomal locations having specific recognition sites (O'Gorman et al. (1991) Science 251:1351; Onouchi et al. (1991) Nucleic Acids Res. 19:6373; Logie and Stewart (1995) Proc. Natl. Acad. Sci. USA 92:5940-5944; Shang et al. (1996) Nucleic Acids Res. 24:543-548; Nichols et al.(1997) Mol. Endocrinol. 11:950-961; Feil et al. (1997) Biochem. Biophy. Res. Comm. 237:752-757; Albert et al. (1995) Plant J 7:649-659; Schlake and Bode (1994) Biochemistry 33:12746-12751; O'Gorman et al. (1997) Proc. Natl. Acad. Sci. USA 94:14602-14607; and Araki et al. (1997) Nucleic Acids Res. 25:868-872. However, this approach has its own drawbacks. It requires both the presence of specific target sequences in the host genome, which generally must be previously engineered within desired chromosomal target sites, as well as specific recombinases that must be supplied to the host cell, such as with host cell expression of a recombinase transgene.

Transposable genetic elements or transposons provide another tool for genetic manipulation of organisms. Transposons are DNA sequences that can move or transpose from one position to another position in a genome. These movable elements are found in a wide variety of prokaryotic and eukaryotic organisms. In vivo, intra-chromosomal transpositions as well as transpositions between chromosomal and non-chromosomal genetic material are known. In several systems, transposition is known to be under the control of a transposase enzyme that is typically encoded by the transposable element. The genetic structures and transposition mechanisms of various transposable elements are summarized, for example, in “Transposable Genetic Elements” in The Encyclopedia of Molecular Biology, ed. Kendrew and Lawrence (Blackwell Science, Ltd., Oxford, 1994), incorporated herein by reference.

Transposable elements normally comprise a gene encoding the transposase protein and a so-called transposable cassette, which comprises a region of DNA flanked by end sequences that are recognized by the transposase protein. Transposase protein produces integration of the transposable cassette into the genome of a host cell via recognition of and interaction with the flanking end sequences of the transposable cassette. Subsequent insertion into the genome of the host cell occurs randomly or at “hot spot” sites. In wild-type transposons, the transposase gene may reside within the transposable cassette. This provides a means for subsequent movement of the transposon following insertion within a host genome. For purposes of genetic engineering, further migration of an inserted transposon can be eliminated by repositioning the transposase gene outside the transposable cassette.

While transposons have been used extensively for mutagenesis and cloning in bacteria, and less so in eukaryotic organisms, their random insertion within a host cell genome makes targeted genetic manipulation difficult. Furthermore, the need for an active transposase protein, and in some instances additional accessory proteins, to assist with integration requires that the engineered host cell be provided with these accessory proteins to achieve this random integration.

Even with the advances in genetic modification of host organisms, the major problems associated with conventional gene transformation techniques have remained essentially unresolved as to the problems discussed above relating to random insertion and variable expression levels due to chromosomal position effects and copy number variation of transferred genes. For these reasons, efficient methods are needed for targeting and control of insertion of nucleotide sequences to be integrated into a host's genome.

### SUMMARY OF THE INVENTION

Compositions and methods for targeted genetic manipulation of an organism are provided. The compositions are novel integration vectors derived from the Mu bacteriophage. These integration vectors comprise a Mu cleaved donor complex (CDC) and a “navigator element” that provides for transposition of the Mu transposable cassette in a site-specific manner and in the absence of the accessory protein MuB. Methods of the invention utilize the novel integration vectors of the invention to genetically modify the genome of an organism. Transformation of a host organism with an integration vector of the invention results in insertion of the entire sequence of the Mu transposable cassette into a predetermined target site in the organism's genome. The Mu transposable cassette may contain any sequences of interest, including one or more regulatory and/or coding sequences. Sequences of interest include sequences encoding selectable markers, drug resistance markers, or other genes, for example, genes promoting agronomically useful traits in crop plants. The methods of the invention are useful for modulating activity of known genes and for targeting integration of nucleotide sequences of interest into a specific location of an organism's genome. The methods are also useful in creating deletions of genes or regulatory regions in a genome.

In some embodiments of the integration vectors of the invention, cleaved donor complexes (“CDCs”) are derived from a precleaved mini-Mu plasmid and are attached to one or more “navigator elements” as part of the targeting mechanism. A navigator element attached to the CDC facilitates localization of the Mu transposable cassette to a predetermined binding site within the genome of an organism of interest and thereby promotes integration of the Mu transposable cassette into a predetermined target site in the genome. The CDC may be prepared from a precleaved mini-Mu plasmid or other DNA derived from bacteriophage Mu. In some embodiments of the integration vector of the invention, the navigator element is a single-stranded DNA sequence sharing homology with a predetermined binding sequence adjacent to the host target site for insertion of the Mu transposable cassette. In such embodiments, the single-stranded DNA navigator-attached CDC may be incubated with a RecA-like protein to obtain a RecA-coated single-stranded CDC integration vector. In such embodiments, RecA-like protein may be noncovalently bound to the flanking single-stranded non-Mu plasmid DNA sequences. When a host cell is transformed with this integration vector, the flanking RecA-coated single-stranded DNA sequence comprising the region of homology hybridizes to the predetermined binding sequence adjacent to the predetermined target site within the host organism's genome. Following this RecA-assisted hybridization, the MuA protein bound as part of the single-stranded CDC facilitates transfer of the entire Mu transposable cassette into the target site of the organism's genome.

Various navigator elements may be used in the compositions and methods of the invention. Navigator elements may be composed of DNA, RNA, protein, peptide nucleic acid (PNA) or other chemical entity or any combination thereof so long as the navigator element is capable of providing localization to a predetermined binding site in the genome.

### BRIEF DESCRIPTION OF THE DRAWINGS

FIG. 1 schematically depicts the key features of the transposable region of the bacteriophage Mu, which serves as the basis for the key features of the wild-type mini-Mu plasmid. The Mu left end recognition sequence, which comprises the attL1 (L1), attL2 (L2), and attL3 (L3) end-type MuA transposase binding sites, the Mu right end recognition sequence, which comprises the attR1 (R1), attR2 (R2), and attR3 (R3) end-type MuA transposase binding sites, the transpositional enhancer sequence (also referred to herein as the internal activating sequence, IAS), the MuA transposase sequence, and the MuB sequence are shown. In the wild-type mini-Mu plasmid, the transposable cassette region (left end and right end recognition sequences flanking an internal nucleotide sequence comprising the IAS) is flanked by the non-Mu plasmid DNA domain (narrow solid black line).

FIG. 2 schematically depicts the normal molecular mechanism of Mu transposition. In the presence of MuB, intermolecular transposition occurs; in its absence, transposition is predominately intramolecular.

FIG. 3 schematically depicts Mu donor cleavage and strand transfer to a target site. L=left end recognition sequence; R=right end recognition sequence.

FIG. 4 schematically depicts formation of the Mu phage-derived cleaved donor complex (CDC) by addition of MuA and Mg2+ to Mu DNA.

FIG. 5 schematically depicts generation of a CDC with an ssDNA navigator element generated with an Exonuclease III (“ExoIII”) digestion step.

FIG. 6 schematically depicts coating of a single-stranded region of an ssCDC with RecA-like protein, forming a RecA-coated ssCDC, which is shown annealed to the target genome site. Stars indicate the position of insertion.

FIG. 7 schematically depicts preparation of a precleaved mini-Mu plasmid (“precleaved Mu”) as compared to DNA cleavage of the wild-type (WT) mini-Mu plasmid. To prepare an active CDC, the precleaved mini-Mu plasmid would be incubated with MuA transposase. L=left end recognition sequence; R=right end recognition sequence.

FIG. 8 schematically depicts attachment of a navigator element comprising single-stranded DNA to a precleaved mini-Mu plasmid prior to addition of MuA to form an active CDC by addition of MuA.

FIG. 9 schematically depicts preparation of a navigator-attached CDC. In the embodiment depicted in this Figure, the navigator used is single-stranded DNA. Note that the navigator could be, for example, a protein or a chemical navigator, and the procedure to attach the navigator could be, for example, an enzymatic or chemical reaction.

FIG. 10 schematically depicts the use of homology-assisted transposition with a navigator-attached CDC. As indicated in the Figure, homology-assisted transposition can occur in vitro and can be used to create a knockout of any target gene.

FIG. 11 schematically depicts homology-assisted transposition into a target site of the host genome using navigator-attached CDCs. As shown in the Figure, embodiments include navigator elements composed of single-stranded DNA or peptide nucleic acid. Embodiments also include “affinity-assisted transposition” using a molecular probe as a navigator element.

FIG. 12 schematically depicts reactions after hybridization of RecA-coated ssCDC with the target site in the organism's genome. A) depicts alignment of the RecA-coated ssCDC with the target genome site, and asterisks depict sites of 3′ OH nucleophilic attack on the phosphodiester bonds on the backbone of the target DNA; B) depicts subsequent insertion of the Mu transposable cassette into the target genome site.

FIG. 13 schematically depicts an in vitro gene-targeting assay using a navigator-attached CDC having a single-stranded DNA (ss-DNA) as a navigator element. Plasmid PC-2R contains two sets of “R” recognition sites from Mu bacteriophage, comprising the R1, R2, and R3 sites. Plasmid PC-2R also contains a chloramphenicol resistance gene, designated “CAM,” a Mu Internal Activating Sequence, or “IAS,” and an ampicillin resistance gene, designated “AMP.”

FIG. 14 shows a diagram of plasmid PC-4, a representative plasmid of the invention, which comprises DNA elements capable of forming a CDC complex: MuA binding sites R1, R2, and R3 positioned at the ends of a DNA fragment in inverted orientation. While PC-4 contains a selectable marker for use in bacteria, the use of a selectable marker is not essential to the present invention; in some embodiments, cotransformation of a selectable marker with a CDC and/or screening for transformants are performed.

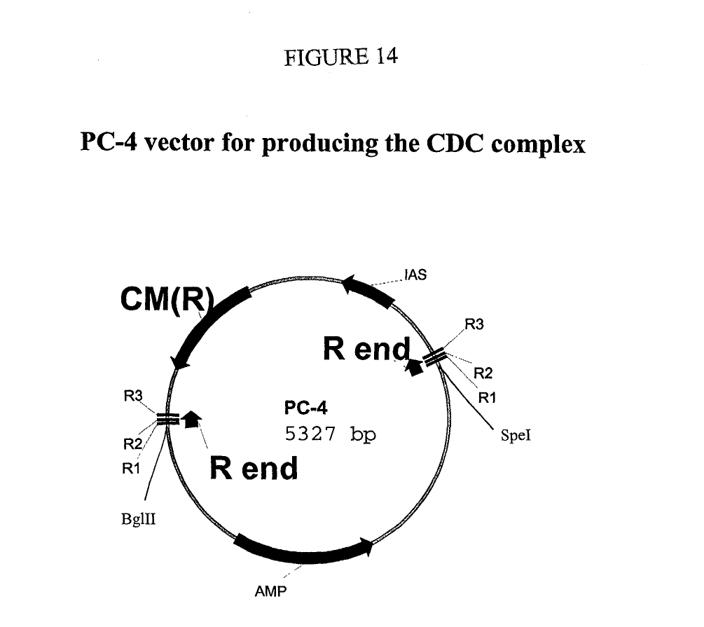

FIG. 15 shows a diagram of representative plasmids of the invention PC-1, PC-2, and PC-3. Each plasmid contains the MuA binding sites R1, R2 and R3 in inverted orientation.

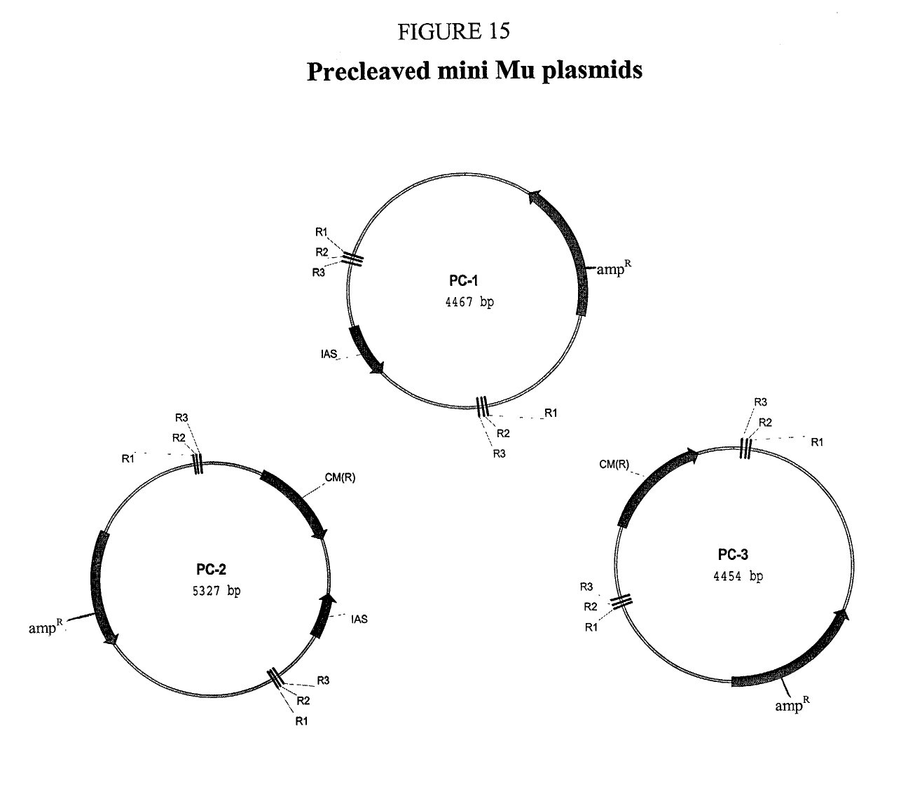

FIG. 16 shows a diagram of an in vitro targeting experiment in which the ssDNA portion of the navigator element was attached to the CDC portion of the integration vector via a biotin/streptavidin portion of the navigator element.

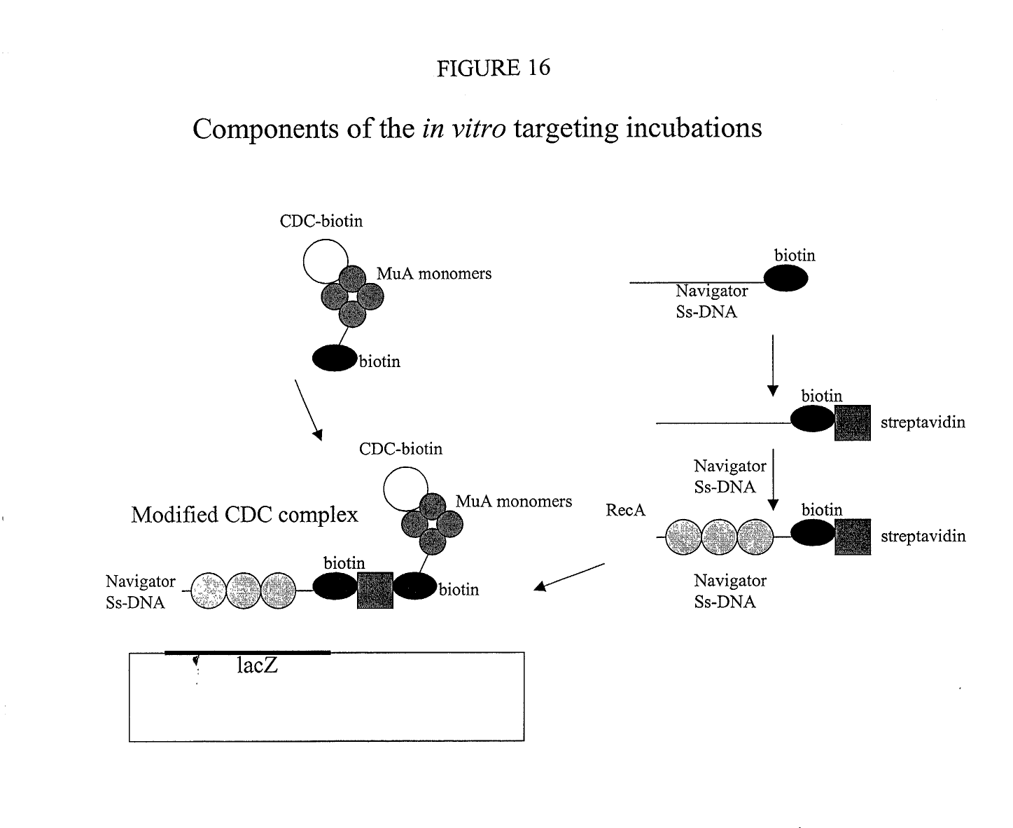

FIG. 17 shows a diagram of experimental strategy and results from an in vitro transposition assay. The experiment demonstrates that transposition occurs in the presence of transposon DNA and MuA protein.

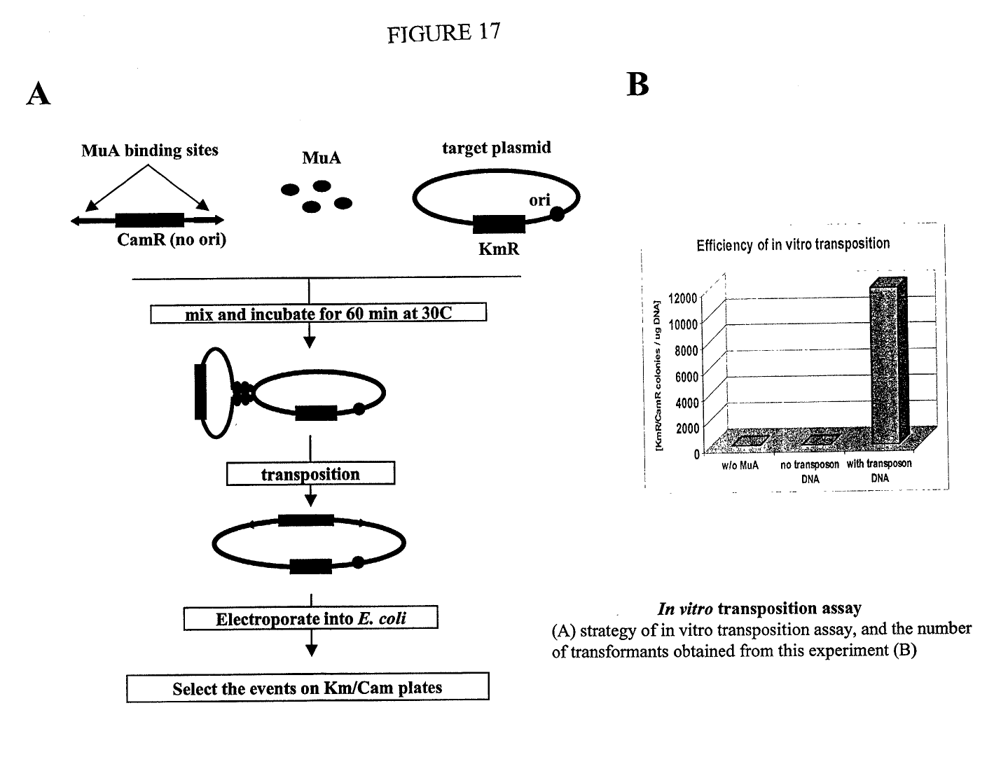

FIG. 18 shows a diagram of experimental strategy and results from an in vivo transposition assay. In this experiment, the formation of active CDCs occurs in the host cell in which MuA is being expressed. The efficiency of in vivo transposition is highest with a high (induced) level of MuA expression.

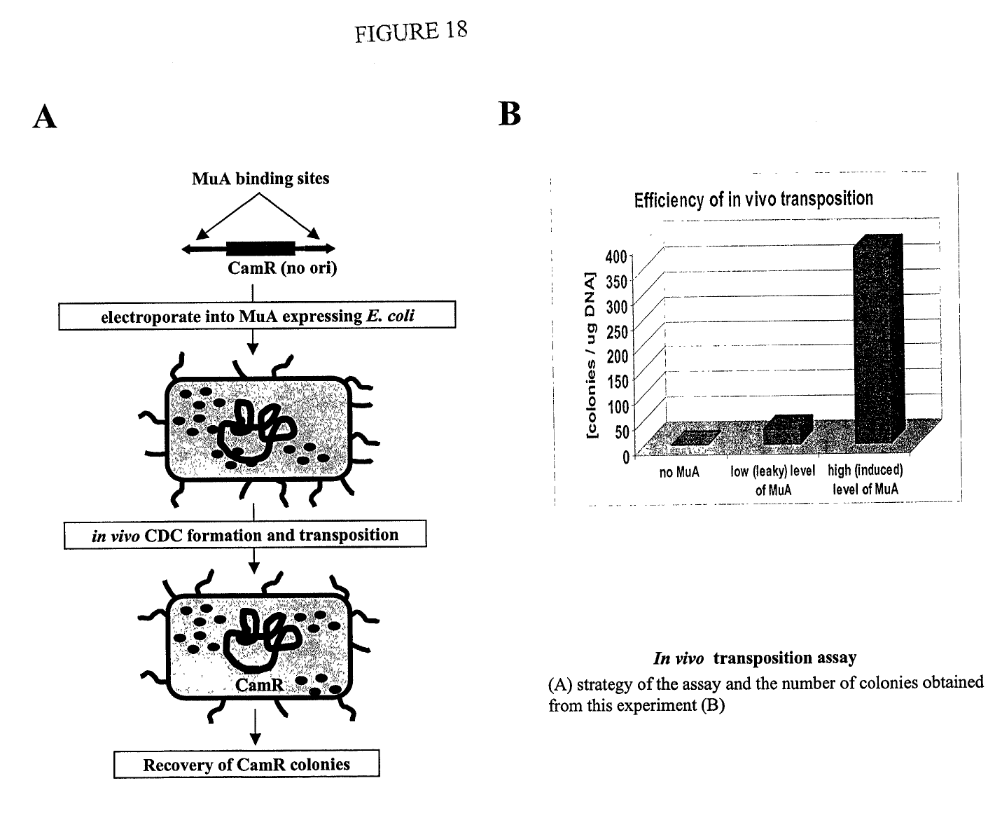

### DETAILED DESCRIPTION OF THE INVENTION

The present invention is directed to compositions and methods for targeted genetic manipulation of an organism, more specifically for insertion of a transposable cassette into a predetermined target site within an organism's genome. Those of skill in the art will appreciate that multiple genes and any regulatory sequences necessary for appropriate gene expression may be included in the transposable cassette. Insertion of the transposable cassette results in altered gene expression within the host organism. Such altered gene expression includes, but is not limited to: decreased or enhanced expression of an endogenous gene to manipulate the level of endogenous gene product; newly created expression of a foreign nucleotide sequence to facilitate physiological and/or phenotypic manipulation of an organism; disruption of expression of an endogenous gene, including a “knockout” or destruction of gene function of the disrupted gene; and disrupting expression of an endogenous gene while promoting expression of a variant of the disrupted endogenous gene product, where expression of the variant gene product confers a desirable attribute to the host organism. An “endogenous gene” may be a gene that is naturally occurring within the organism or a transgene that has previously been integrated into the organism's genome.

Compositions of the invention are novel integration vectors that are derived from cleaved donor complexes (CDCs) of the temperate bacteriophage Mu, a bacterial class III transposon of Escherichia coli. This transposon exhibits extremely high transposition frequency (Toussaint and Résibois (1983) in Mobile Genetic Elements, ed. Shapiro (Academic Press, New York), pp. 105-158). The Mu bacteriophage with its approximately 37 kb genome is relatively large compared to other transposons. Mu encodes two gene products that are involved in the transposition process: MuA transposase, a 70 kDa, 663 amino-acid multidomain protein, and MuB, an accessory protein of approximately 33 kDa. This transposable element has left end and right end MuA recognition sequences (designated “L” and “R”, respectively) that flank the Mu transposable cassette, the region of the transposon that is ultimately integrated into the target site. Unlike other transposons known in the art, these ends are not inverted repeat sequences. The Mu transposable cassette, when necessary, may include a transpositional enhancer sequence (also referred to herein as the internal activating sequence, or “IAS”) located approximately 950 base pairs inward from the left end recognition sequence.

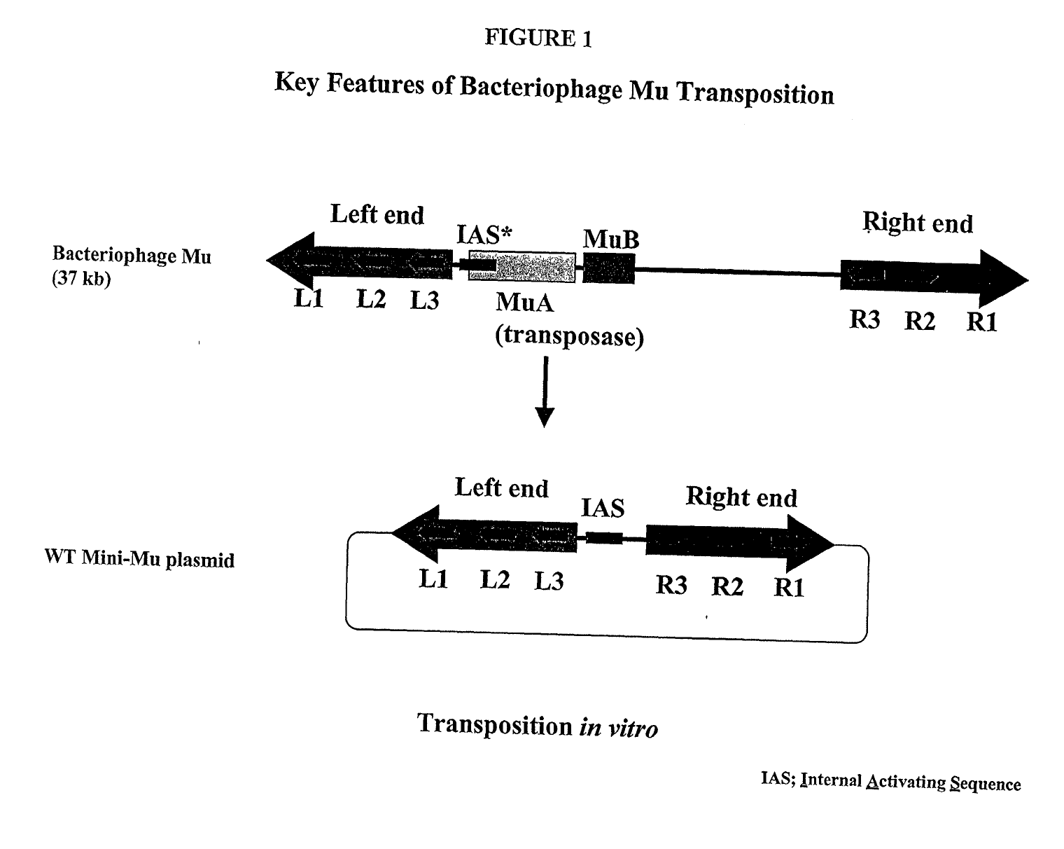

The left and right end recognition sequences of the Mu transposon each encompass three 22-base-pair “end-type” MuA transposase binding sites, designated attL1 (“L1”), attL2 (“L2”), and attL3 (“L3”); and attR1 (“R1”), attR2 (“R2”), and attR3 (“R3”), which are numbered from the extreme ends of the Mu transposable cassette inwards (see FIG. 1). Two dinucleotide DNA cleavage sites reside outside the Mu transposable cassette, positioned 6 bp away from the end-most MuA-binding sites L1 and R1. The Mu transpositional enhancer sequence also binds the MuA transposase, but at a different domain of the protein than that used to bind the left and right end recognition sequences. MuA transposase interacts with the flanking left and right end recognition sequences and the transpositional enhancer sequence to bring about insertion of the Mu transposable cassette into a target DNA sequence.

Transposition is an essential feature of the life cycle of bacteriophage Mu. Integration of infecting Mu DNA into a host chromosome to form a stable lysogen occurs by nonreplicative simple insertion (Liebart et al. (1982) Proc. Natl. Acad. Sci. USA 79:4362-4366; Harshey (1984) Nature 311:580-581. During lytic growth, Mu generates multiple copies of its genome by repeated rounds of replicative transposition (Ljungquist and Bukhari (1977) Proc. Natl. Acad. Sci. USA 74:3143-3147) via a cointegrate pathway (Chaconas et al. (1981) J. Mol. Biol. 150:341-359). Both types of transposition are facilitated by the MuA transposase and accessory MuB protein. E. coli-encoded proteins such as histone-like protein (“HU”) and integration host factor (IHF) assist in early conformational changes that ultimately lead to the transfer of the Mu transposable cassette into a target host DNA sequence.

The details of Mu transposition have been elucidated using an in vitro transposition reaction (Mizuuchi (1983) Cell 35:785-794; Mizuuchi (1984) Cell 39:395-404; Craigie and Mizuuchi (1985) Cell 41:867-876; Craigie et al. (1985) Proc. Natl. Acad. Sci. USA 82:750-7574; reviewed by Chaconas et al. (1996) Curr. Biol. 6:817-820; Craigie (1996) Cell 85:137-140; Lavoie and Chaconas(l995) Curr. Topics Microbiol. Immunol. 204:83-99; and Mizuuchi (1992) Annu. Rev. Biochem. 61:1011-1051). In this in vitro reaction, for example, the transposon donor is a mini-Mu plasmid, and another DNA molecule, commonly φX174 replicative form DNA, serves as the target of transposition. The mini-Mu plasmid is constructed such that it comprises two DNA domains. The first of these DNA domains is a Mu transposable cassette, which is flanked by the second DNA domain, referred to herein as the non-Mu plasmid DNA domain (see FIG. 1).

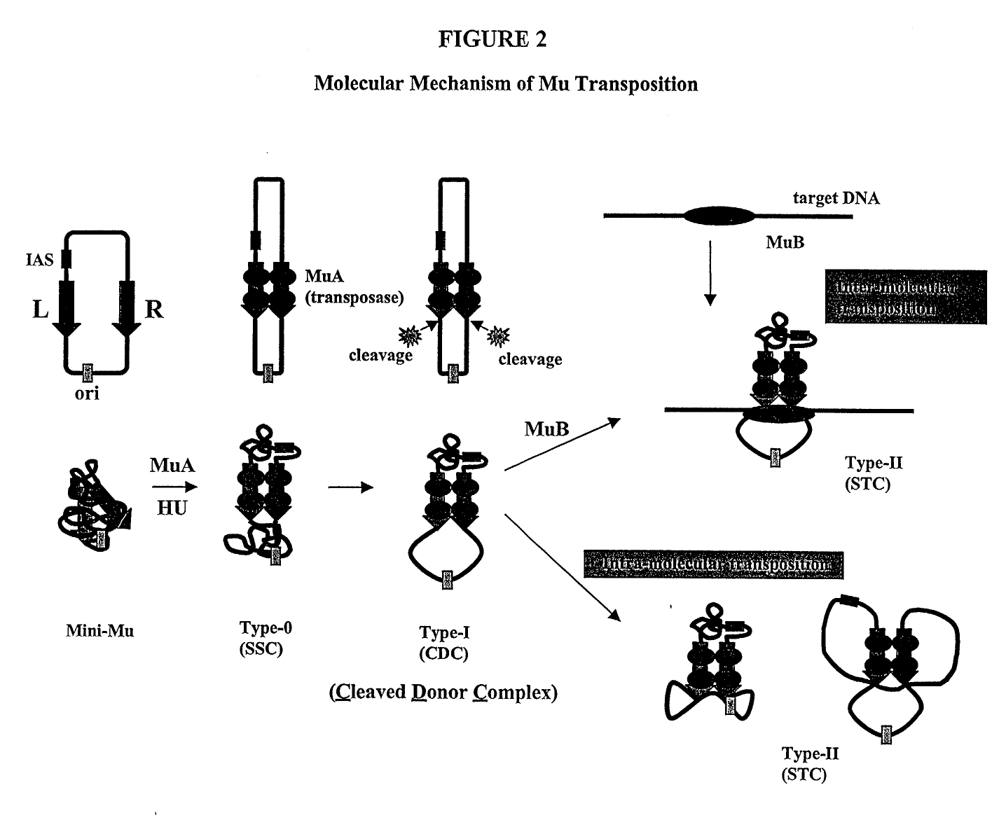

Using an in vitro system, it has been shown that normally MuA transposase exists in its inert monomeric state which does not recognize the DNA cleavage sites adjacent to the left end and right end recognition sequences of the Mu transposable cassette. In the presence of HU, IHF, and divalent metal ions, particularly Mg²⁺, MuA transposase initially binds to the Mu transpositional enhancer sequence and to the left and right end recognition sequences. Following this binding, the mini-Mu plasmid undergoes a series of conformational changes that ultimately result in formation of the cleaved donor complex (CDC). It is believed that in this stable nucleoprotein complex, a single-stranded nick has been introduced at each end of the Mu transposable cassette, exposing 3′ OH groups that act as nucleophiles and attack the target DNA sequence. However, the 5′ ends of the Mu transposable cassette remain attached to the 3′ ends of the non-Mu plasmid DNA (see FIG. 2).

Accordingly, a cleaved donor complex or CDC of the present invention is derived from a plasmid comprising sequences derived from Mu bacteriophage DNA sequences. While the invention is not bound by any theory, this plasmid DNA is treated with MuA so as to induce conformational changes and nick the DNA so that exposed 3′—OH groups can act as nucleophiles and attack a target DNA sequence. Alternatively, the exposed 3′—OH groups may result from treatment with restriction enzymes. An “active” CDC of the present invention may comprise a MuA tetrameric core or the MuA may be removed from the CDC to prepare a “stripped down” CDC, as further discussed below.

In normal bacteriophage Mu transposition, the structural and functional core of the CDC is a tetrameric unit of MuA molecules (Lavoie et al. (1991) EMBO J. 10:3051-3059; Mizuuchi (1992) Annu. Rev. Biochem. 61:1011-1051; Baker et al. (1993) Cell 74:723-733, hereinafter referred to as the MuA tetrameric core. The three end-type MuA transposase binding sites designated attL1, attR1, and attR2 are considered the core binding sites, as they are stably bound by the MuA tetramer. MuA protein interacting with the other three end-type MuA transposase binding sites (attL2, attL3, and attR3) is loosely bound. These loosely bound MuA molecules can be removed either by heparin, high salt (0.5 M NaCl), or excess Mu end competitor DNA (Kuo et al. (1991) EMBO J. 10:1585-1591; Lavoie et al. (1991) EMBO J. 10:3051-3059; Mizuuchi et al. (1991) Proc. Natl. Acad. Sci. USA 88:9031-9035). The three sites L1, L2, and L3 are considered accessory sites, as they are dispensable individually and are not required for the intermolecular strand transfer reaction (Allison and Chaconas (1992) J. Biol. Chem. 267:19963-19970; Lavoie et al. (1991) EMBO J. 10:3051-3059; and Mizuuchi et al. (1991) Proc. Natl. Acad. Sci. USA 88:9031-9035). However, sites R1, R2 and R3 may be interchanged with sites L1, L2, and L3 for use in constructing plasmids and in preparing the active cleaved donor complexes of the invention.

In the in vitro system, as well as in bacterial cells, the Mu-encoded protein MuB binds to target DNA in a non-specific manner in the presence of ATP. Accordingly, in the in vitro system, MuB binds to the target DNA molecule, while in vivo it binds to host DNA. The DNA-bound form of MuB has a strong affinity for the Mu CDC, and thus, when present, MuB introduces the CDC to the target molecule or host genome wherever MuB is bound. Because of the non-specific binding of MuB, CDC introduction occurs with little target preference. MuB also stimulates the DNA-breakage and DNA-joining activities of MuA (Adzuma and Mizuuchi (1988) Cell 53:257-266; Baker et al. (1991) Cell 65:1003-1013; Maxwell et al. (1987) Proc. Natl. Acad. Sci. USA 84:699-703; Surette and Chaconas (1991) J. Biol Chem. 266:17306-17313; Surette et al. (1991) J. Biol. Chem. 266:3118-3124; and Wu and Chaconas (1992) J. Biol. Chem. 267:9552-9558; and Wu and Chaconas, (1994) J. Biol. Chem. 269:28829-28833). Thus, MuBbound DNA molecules are preferential targets of Mu transposition. In the absence of MuB, introduction of the CDC to a target DNA site still occurs but is mainly limited to intramolecular reactions which take place in adjacent regions outside of Mu DNA (see FIG. 2).

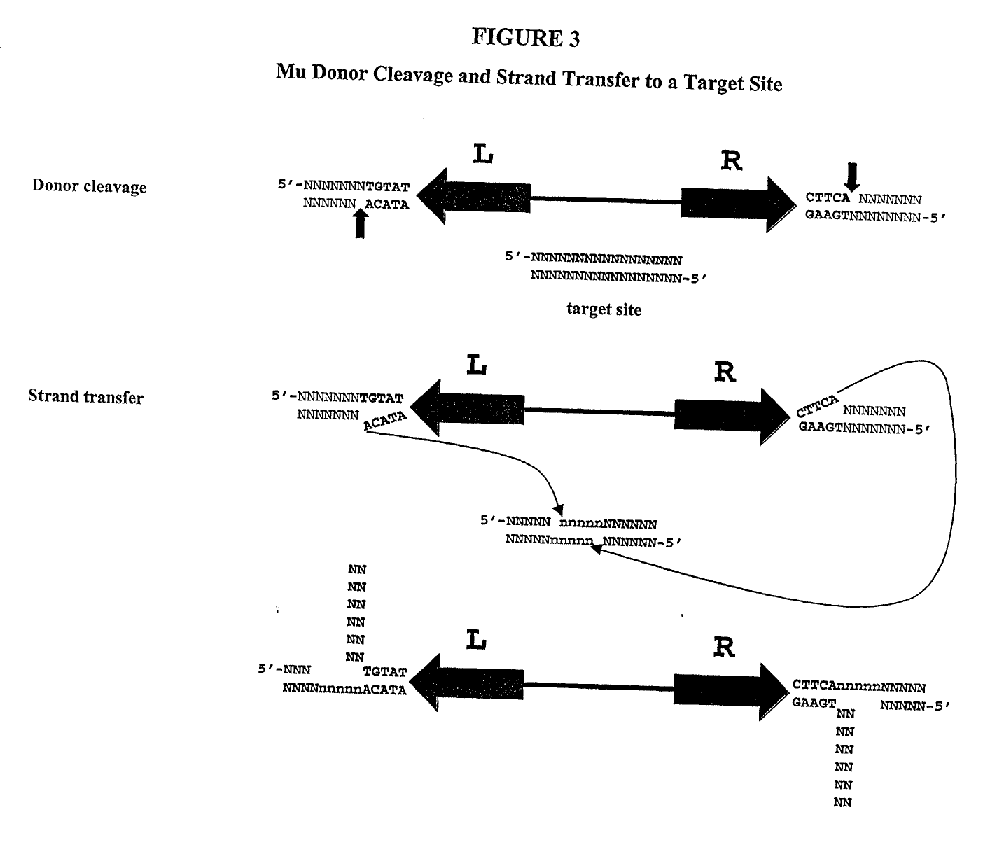

The actual transfer of the Mu transposable cassette from the CDC into a target DNA site is mediated by the bound MuA transposase within the CDC. While the invention is not bound by any theory or mechanism of action, it is believed that the exposed 3′ OH ends of the CDC act as nucleophiles, attacking the phosphodiester bond on the backbone of the target DNA. This attacking of a phosphate group by the exposed 3′ OH group forms a bond between the 3′ ends of the Mu DNA and the 5′ ends of the target DNA (see FIG. 3). This process is referred to as strand transfer and results in formation of a strand transfer complex (STC). This stable nucleoprotein complex is involved in both cointegration and simple insertion (see generally, Haren et al. (1999) Ann. Rev. Microbiol 53:245-281). Cointegrates are made by replication of the Mu transposable cassette portion of the STC, using the free 3′ ends of the target DNA as primers for leading-strand DNA synthesis. Simple inserts are formed from the STC by degradation of the non-Mu plasmid DNA domain that flanked the Mu transposable cassette portion of the donor molecule, followed by gap repair.

The integration vectors of the present invention comprise Mu bacteriophage “active” cleaved donor complexes (CDCs) that have been modified by attachment of navigator elements such that insertion of the Mu transposable cassette within the genome of a host organism occurs in a site-specific manner and in the absence of the accessory protein MuB. This integration can occur in the absence of in vivo expression of MuA transposase because active CDC has the intact MuA tetrameric core attached. These novel integration vectors allow for insertion of the entire Mu transposable cassette within a predetermined target site in any host organism's genome and thus may be referred to as “targeted CDCs.” By “predetermined target site” is intended a desired location within the genome of the host organism for insertion of the Mu transposable cassette. Desired locations in the genome include, for example, locations in chromosomal DNA sequences, episomal sequences (e.g., replicable plasmids or viral replication intermediates), and chloroplast and mitochondrial DNA sequences. By “predetermined” is intended that the target site may be selected by the practitioner on the basis of known or predicted sequence information.

When targeting an insertion within chromosomal DNA, the desired position may be chosen such that the Mu transposable cassette is inserted adjacent to (i.e., either 5′ or 3′ to) or within a known structural gene, promoter, enhancer, recombinatorial hotspot, repeat sequence, integrated proviral sequence, etc. For example, where the integration vector is used to disrupt expression of a particular gene, the predetermined target site will reside within the regulatory region and/or coding sequence for that gene. The predetermined target site may reside in a naturally occurring host cell nucleotide sequence or in a sequence that has been previously engineered or added to the genome. Thus, “predetermined target sites” may include engineered sites recognized by site-specific recombinases such as, for example, CRE recombinase or FLP recombinase. One of skill in the art will appreciate that the desired results may be achieved whether the transposable cassette actually integrates into the predetermined target site or whether it integrates into some other site.

In some embodiments, an integration vector is used to disrupt expression of a particular gene. The term “disrupt” indicates that expression from a gene has been virtually eliminated so as to result in an absence of gene expression from the disrupted gene copy. A “knockout” is a disruption in which the absence of gene expression is a result of partial or complete deletion of the coding and/or regulatory regions of the native gene. Thus, knockouts can result from replacement of the complete coding region of a gene with introduced genetic material.

Where the integration vector is to be used to insert a nucleotide sequence encoding a gene of interest, preferably the predetermined target site is chosen to minimize position effects, thereby allowing expression of the inserted nucleotide sequence in an amount sufficient to produce the desired effect. In one embodiment, the predetermined target site resides within the regulatory region and/or coding sequence of an endogenous gene and the integration vector comprises an expression cassette containing a coding sequence for a protein that is a variant of the protein encoded by the endogenous gene targeted for disruption. Expression of this variant protein confers a desirable attribute to the host organism. Insertion of the integration vector into the gene target allows for disruption of expression of the endogenous gene product and expression of the desired variant protein. Alternatively, endogenous genes could be partially or entirely removed from the genome to create a gene “knockout” or deletion. Also, a series of reactions could be performed leading to insertion of multiple genes, promoter fragments, etc.

Active cleaved donor complexes (CDCs) can be obtained using an in vitro transposition reaction and a mini-Mu plasmid as the transposon donor. By “mini-Mu plasmid” is intended a plasmid comprising a Mu transposable cassette flanked by a nonMU plasmid DNA domain. Such mini-Mu plasmids can be constructed using molecular biology techniques well known in the art. See particularly Sambrook et al. (1989) Molecular Cloning: A Laboratory Manual (2d ed.; Cold Spring Harbor Laboratory Press, Plainview, N.Y.); and Ausubel et al., eds. (1995) Current Protocols in Molecular Biology (Greene Publishing and Wiley-Interscience, New York).

Any plasmid or mini-Mu plasmid can be used to obtain the CDCs, so long as it comprises the necessary elements within the Mu transposable cassette for formation of an active CDC. By “active CDC” is intended a CDC that is capable of carrying out intermolecular or intramolecular strand transfer in an in vitro transposition reaction. Such active CDCs, when modified to obtain the integration vectors of the present invention, will support intermolecular strand transfer in vivo. The necessary elements for active CDC formation depend upon the reaction conditions used during in vitro formation of the CDC (see, for example, Baker and Mizuuchi (1992) Genes and Develop. 6:2221-2232; Wu and Chaconas (1997) J. Mol. Biol. 267:132-141). However, it is possible to obtain an active CDC using a Mu transposable cassette the ends of which are defined by either the left or right MuA recognition sequences. Further, if precleaved cassettes are used, it is possible to obtain integration into the genome (i.e., an active CDC) which retains less than the full set of three binding sites of either the left or right MuA recognition sequence(s).

Thus, in one embodiment of the invention, an active CDC is obtained using a wild-type mini-Mu plasmid. By “wild-type mini-Mu plasmid” is intended the mini-Mu plasmid has a Mu transposable cassette that comprises the complete Mu left and right end recognition sequences in their natural (i.e., inverted) orientation; these recognition sequences flank an internal nucleotide sequence comprising the Mu transpositional enhancer sequence. By “complete Mu left and right end recognition sequences” is intended each of the end recognition sequences comprising the three naturally occurring 22-base-pair end-type MuA transposase binding sites. Thus, the left end recognition sequence comprises the attL1, attL2, attL3 end-type MuA transposase binding sites, while the right end recognition sequence comprises the attR1, attR2, and attR3 end-type MuA transposase binding sites. When present, the complete end recognition sequences allow for formation of an active CDC having MuA transposase stably bound to the core binding sites attL1, attR1, and attR2 to form the MuA tetrameric core, and MuA transposase monomers loosely bound to the accessory end-type MuA transposase binding sites attL2, attL3, and attR3. The base pair sequences for the complete Mu left and right end recognition sequences and the Mu transpositional enhancer are known in the art. See Kahmann and Kamp (1979) Nature 280:247-250 and Allet (1978) Nature 274:553-558 for the Mu left end and right end recognition sequences; note, however, that both of these references contain sequencing errors. The correct sequence is found in Genbank Accession No. AF083977 (bacteriophage Mu sequence, contributed by Grimaud (Virology 217: 200-210 (1996) and Morgan et al., direct submission (Aug. 13, 1998)). See also, Mizuuchi and Mizuuchi (1989) Cell 58:399-408 for the Mu transpositional enhancer sequence, herein incorporated by reference. However, one of skill in the art will realize that the exact nucleotide sequence of these recognition sequences may vary slightly, and there is not an exact sequence requirement for individual binding domains. Thus, for example, the left end recognition sequence comprises three end-type MuA transposase binding sites that reside within nucleotides 1-180 of Genbank Accession No. AF083977, and the right end recognition sequence comprises three end-type MuA transposase binding sites that reside within nucleotides 36641-36662 of Genbank Accession No. AF083977. In one embodiment of the invention, the MuA transposase binding sites in the left end recognition sequence are represented by nucleotides 6-27 (attL1), 111-132 (attL2), and 151-172 (attL3), respectively, of Genbank Accession No. AF083977; and the MuA transposase binding sites in the right end recognition sequence are represented by nucleotides 36691-36712 (attR1), 36669-36690 (attR2), and 36641-36662 (attR3), respectively, of Genbank Accession No. AF083977. One of skill will realize that variations of these sequences may be employed in the invention so long as the desired result is achieved. Thus, sequences having at least 65%, 70%, 75%, 80%, 85%, 90%, 91%, 92%, 93%, 94%, 95%, 96%, 97%, 98%, 99% or more sequence identity to the native Mu sequences may be employed.

Use of a wild-type mini-Mu plasmid to form an active CDC allows for the in vitro transposition reaction to be carried out under standard reaction conditions. For standard reaction conditions, see Mizuuchi et al. (1992) Cell 70:303-311 and Surette and Chaconas (1992) Cell 68:1101-1108, herein incorporated by reference. When a wild-type mini-Mu plasmid is used in the in vitro transposition reaction under standard conditions, the mini-Mu plasmid must be negatively supercoiled to form an active CDC. However, this requirement for supercoiling under standard reaction conditions can be relieved under other reaction conditions, for example, by including DMSO in the reaction mixture. See Baker and Mizuuchi (1992) Genes and Develop. 6:2221-2232, herein incorporated by reference.

In another embodiment of the invention, an active CDC is obtained using a derivative mini-Mu plasmid. By “derivative mini-Mu plasmid” is intended a mini-Mu plasmid having a Mu transposable cassette that lacks one or more of the features of the Mu transposable cassette found in a wild-type mini-Mu plasmid. By “features” is intended the following: (1) a complete left end recognition sequence, (2) a complete right end recognition sequence, (3) left and right end recognition sequences in their natural orientation (i.e., inverted), and (4) a Mu transpositional enhancer sequence within the internal nucleotide sequence that is flanked by the left and right end recognition sequences. Thus, for example, a derivative mini-Mu plasmid lacking a complete left or right end recognition sequence lacks one or more of the end-type MuA transposase binding sites within its Mu transposable cassette.

Where a derivative mini-Mu plasmid is used to obtain an active CDC, the reaction conditions required in an in vitro transposition reaction will depend upon what wild-type mini-Mu plasmid feature is missing from the Mu transposable cassette. Thus, where the only feature missing is the accessory end-type MuA transposase binding site attR3, standard reaction conditions will yield an active CDC that supports intermolecular strand transfer (Baker and Mizuuchi (1992) Genes and Develop. 6:2221-2232).

Other derivative mini-Mu plasmids having additional features deleted from the Mu transposable cassette can be used to obtain an active CDC by varying the in vitro reaction conditions. For example, when dimethylsulfoxide (DMSO) is included in the transposition reaction under standard reaction conditions, mini-Mu plasmids lacking the Mu transpositional enhancer, carrying only a complete Mu left end or right end recognition sequence, carrying only a single end-type MuA transposase binding site adjacent to a DNA cleavage site with or without the Mu transpositional enhancer, or having left and right end recognition sequences in direct orientation (rather than inverted orientation) can be used to form a CDC that is active in the DNA cleavage and strand transfer steps required for intermolecular transposition. See Baker and Mizuuchi (1992) Genes and Develop. 6:2221-2232, herein incorporated by reference. In the embodiments of the invention, the DNA cleavage site can be a site which is recognized and cleaved by the MuA protein, or it may be a site which is a restriction enzyme recognition site; thus, the DNA cleavage sites used in embodiments of the invention may be native to the DNA sequence in which they are located or they may be engineered or added artificially to the sequence in which they are located.

Accordingly, any plasmid or mini-Mu plasmid that yields an active CDC may be used as the basis for obtaining the integration vectors of the invention. Examples of wild-type mini-Mu plasmids that may be used include, but are not limited to, the pBR322-based pBL07 (7.2 kb; Lavoie (1993) in Structural Aspects of the Mu Transpososome (University of Western Ontario, London, Canada); pUC19-based pBL03 (6.5 kb; Lavoie and Chaconas (1993) Genes Dev. 7:2510-2519; pMK586 (Mizuuchi et al. (1991) Proc. Natl. Acad. Sci. USA 88:9031-9035); pMK108 (Mizuuchi (1983) Cell 35:785-794; Craigie and Mizuuchi (1986) Cell 45:793-800; pCL222 (Chaconas et al. (1981) Gene 13:37-46); and pBR322-based pGG215 (7.1 kb; Surette et al. (1987) Cell 49:253-262). Examples of derivative mini-Mu plasmids having one or more MuA binding sites and/or the transpositional enhancer sequence include, but are not limited to, pBL05 (MuA transposase binding site attR3 deleted from pBL03; Allison and Chaconas (1992) J. Biol. Chem. 267:19963-19970); pMK426 (carrying two Mu right end recognition sequences; Craigie and Mizuuchi (1987) Cell 51:493-501); pMK412 (pMK108 with the Mu transpositional enhancer sequence removed; Mizuuchi and Mizuuchi (1989) Cell 58:399-408); and pMK395 (mini-Mu with wrong relative orientation of the two Mu end sequences; Craigie and Mizuuchi (1986) Cell 45:793-800; and others described in Mizuuchi and Mizuuchi (1989) Cell 58:399-408, herein incorporated by reference. Also suitable for formation of an active mutant CDC are pUC19 derivatives carrying specific MuA-binding sites, such as the derivatives described by Baker and Mizuuchi et al. (1992) Genes and Develop. 6:2221-2232. All of the foregoing references describing such mini-Mu plasmids are herein incorporated by reference.

To obtain the novel targeted integration vectors of the invention, a mini-Mu plasmid comprising the Mu transposable cassette may be engineered to further comprise and/or interact with components of a targeting mechanism that serves to direct the integration of the Mu transposable cassette into a particular site. The targeting mechanism may be provided by any combination of components attachable to the CDC or that are useful in directing the integration of the Mu transposable cassette to a particular site or enhancing the efficiency of integration. Where a component of the targeting mechanism is attached or bound to the CDC, it may be referred to as a “navigator element”. Components of the targeting mechanism may include elements or aspects of the DNA or other molecule into which integration is desired, for example, a region of sequence in the target DNA to which a navigator element (for example, single-stranded DNA) shares homology.

Thus, in some embodiments of the invention, a mini-Mu plasmid comprising the Mu transposable cassette is engineered to comprise a navigator element within the non-Mu plasmid DNA domain prior to formation of the active cleaved donor complex. This navigator element comprises a region of DNA that shares homology with a predetermined binding sequence within a host organism's genome. By “predetermined binding sequence” or “predetermined binding site” (terms used interchangeably herein) is intended a nucleotide sequence within the host organism that is adjacent to (i.e., either 5′ or 3′ to) a predetermined target site within the organism's genome. Depending upon the location of the predetermined target site, the predetermined binding sequence may reside within a chromosomal, mitochondrial, chloroplast, viral, episomal, or mycoplasmal nucleotide sequence. Further, the predetermined binding sequence may exist as a single copy, such as a binding sequence within a single-copy gene serving as the predetermined target site. Alternatively, multiple copies of the predetermined binding sequence may exist within the genome, such as when the predetermined binding site is within a multi-copy gene within the organism's genome. The predetermined binding sequence within the genome of the host organism serves as a reference sequence for constructing the region of homology within the non-Mu plasmid DNA domain of the mini-Mu plasmid. The predetermined binding sequence is at least about 10, 15, 25 nucleotides, or about 50 or 100 nucleotides, or about 200, 250, 300, 500 to about 700, 800, 900, or 1000 nucleotides, up to about 2000 to 5000 nucleotides in length. In embodiments in which the navigator element is composed of nucleic acid or nucleic acid analog, such as DNA, RNA, or PNA, the localization of the CDC to the target site may depend on sequence identity between the navigator element and the target site. In such embodiments, the region of homology within, for example, the navigator will share at least about 70 percent sequence identity, or at least about 80 percent sequence identity, or at least about 85 percent sequence identity, or at least about 90, 95, 96, 97, 98, 99, or 100 percent sequence identity with the DNA strand complementary to the predetermined binding sequence. The percentage of sequence identity is calculated excluding small deletions or additions which total less than 25 percent of the predetermined binding sequence. Methods for determining sequence identity are known in the art (see particularly the discussion elsewhere herein). In some embodiments, the non-Mu plasmid DNA domain of the mini-Mu plasmid has a region of homology that is at least about 25 to 35 nucleotides long, preferably at least about 50 to 100 nucleotides long, more preferably at least about 100 to about 500 nucleotides long, up to about 1000, 2000, 3000 nucleotides long, although the degree of sequence identity between the region of homology and the predetermined binding sequence or its complementary strand, as well as the base composition of the predetermined binding sequence, will determine the optimal and minimal lengths for the region of homology. For example, G-C rich sequences are typically more thermodynamically stable and will generally require shorter regions of homology within the non-Mu plasmid DNA domain. Therefore, the minimum requirements for both the length of the region of homology and the degree of sequence homology to the predetermined binding sequence can only be determined with respect to a particular predetermined binding sequence. Generally, the region of homology within the non-Mu plasmid DNA domain must be at least about 25 nucleotides long and must also share at least about 70% sequence identity with the complementary strand of predetermined binding sequence in the host organism's genome. Preferably, the region of homology is at least about 25 nucleotides long and is at least about 95% identical to the complementary strand of predetermined binding sequence.

Where in vitro production of active CDCs is desired, the resulting mini-Mu plasmid is then subjected to the initial steps of the in vitro transposition reaction to form an active cleaved donor complex (CDC). Methods for producing active CDCs are well known in the art. See particularly Craigie et al. (1985) Proc. NatL. Acad. Sci. USA 82:7570-7574; Wu and Chaconas (1997) J. Mol. Biol. 267:132-141, herein incorporated by reference. The transposition reaction may be carried out under standard reaction conditions (Craigie et al. (1985) Proc. Natl. Acad. Sci. USA 82:7570-7574, herein incorporated by reference) or under modified reaction conditions (such as with the addition of DMSO or glycerol; see, for example, Mizuuchi and Mizuuchi (1989) Cell 58:399-408, herein incorporated by reference) to obtain an active CDC.

Active CDCs may be obtained in vivo (i.e., in the host cell) where MuA is introduced into or expressed in a cell in which DNA from a mini-Mu plasmid or other plasmid capable of forming an active CDC is also present. In some embodiments, for example, formation of active CDCs from DNA of a mini-Mu plasmid previously integrated into the genome of the host organism could result in deletion of most of the previously integrated DNA and could also result in reintegration of the newly-formed active CDC into a different location of the host genome.

For example, where in vitro production of active CDCs is desired, a mini-Mu plasmid of interest is incubated with the purified native MuA transposase protein and the E. coli HU protein, or biologically active variants or fragments thereof as defined below, in the presence of a divalent metal ion such as Mg²⁺ or Mn²⁺ (Mizuuchi et al. 1992 Cell 70:303-311). Where the Mu transposable cassette comprises a Mu transpositional enhancer sequence, the purified E. coli protein IHF or variant thereof is also included in the incubation reaction. Following formation of the CDC, the reaction is terminated by addition of EDTA (see Wu and Chaconas (1997) J. Mol. Biol. 267:132-141) to obtain the stable active CDC. Further spontaneous rearrangements of the CDC can also be inhibited by incubation at 0° C. (see Surette et al. (1987) Cell 49:253-262)). Where the CDC has been derived from a wild-type mini-Mu plasmid, the loosely bound MuA transposase molecules may be removed to obtain a stripped-down version of the active CDC (Wu and Chaconas (1997) J. Mol. Biol. 267:132-141). This stripped-down active CDC may be used for preparing the integration vectors of the invention. However, when the active CDC comprises the MuA transposase molecules loosely bound to the accessory binding sites attL2, attL3, and attR3, intermolecular strand transfer occurs four times faster than with the stripped-down CDC (Wu and Chaconas (1997), supra). Thus, when a stripped-down CDC is to be used, additional MuA protein can be codelivered into the host cell to promoter intermolecular strand transfer. Additional MuA can be codelivered directly using a technique such as microinjection or particle bombardment, or it can be codelivered indirectly by delivering an expression vector comprising the MuA coding sequence operably linked to regulatory elements that promote expression in the host cell. Since MuA must be imported into the nucleus, such a DNA construct would further comprise a sequence encoding a nuclear localization signal, such as the SV40 NLS, fused in frame with the MuA coding sequence. In addition to MuA, other proteins or compounds may be helpful in achieving the desired results of increased frequency of non-random integration of the CDC, and such proteins or compounds may also be codelivered into the host cell with the vectors of the present invention or may be bound to the CDC and/or navigator element.

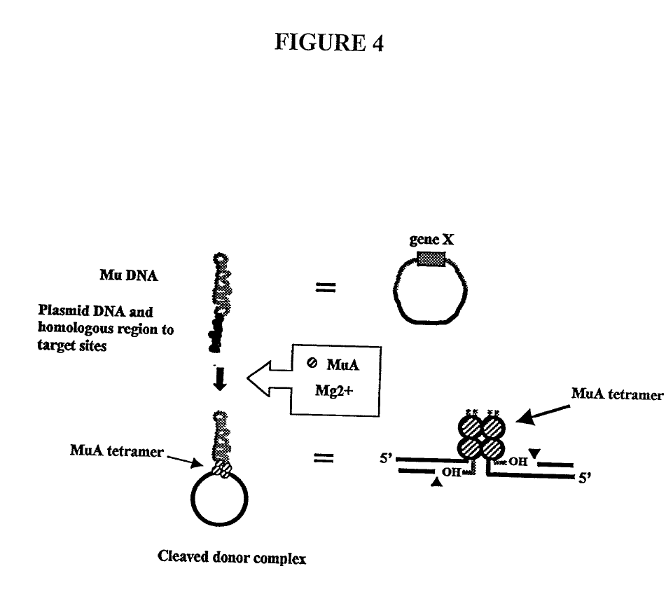

Thus, a mini-Mu plasmid of interest and the native MuA transposase, HU, and IHF proteins, or biologically active variants or fragments thereof, may be used in an in vitro reaction under standard or modified reaction conditions to obtain a stable active CDC that is capable of intermolecular transposition (see FIG. 4). During formation of this CDC, a nick has been introduced at each end of the Mu transposable cassette, exposing 3′—OH groups, relaxing the non-Mu plasmid DNA domain of the mini-Mu plasmid (see FIGS. 2 and 3). This stable CDC may then be modified within the non-Mu plasmid DNA domain to obtain novel integration vectors of the invention.

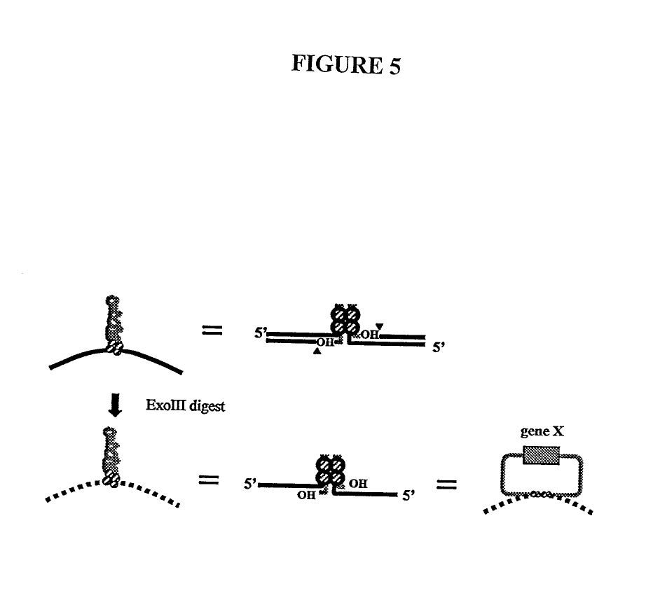

In this manner, one embodiment of the invention provides that the double-stranded non-Mu plasmid DNA sequence is first linearized using a restriction enzyme such as EcoRI to bring about a double-stranded cleavage. This generates two double-stranded non-Mu plasmid DNA sequences that flank the tetrameric core of the original CDC (see FIG. 5). At least one of these flanking double-stranded non-Mu plasmid DNA sequences comprises a region of DNA that shares homology with the complementary strand of the predetermined binding sequence that is adjacent to a predetermined target site for integration into the host genome.

This DNA-protein complex then may be further modified to optimize the targeting mechanism whereby a targeted CDC is provided. For example, this DNA-protein complex may be further modified to comprise at least one single-stranded DNA region which is adjacent to one of the two exposed 3′ OH nucleophilic groups of the tetrameric core of the CDC. In some embodiments, at least one of the double-stranded non-Mu plasmid DNA sequences is digested with the ExoIII exonuclease (see FIG. 5). This enzyme digests DNA only in the 3′ to 5′ direction, generating at least one single-stranded non-Mu plasmid DNA sequence flanking the CDC. Thus, in some embodiments, this single-stranded sequence may extend up to the tetrameric core. Where only one of the two flanking double-stranded non-Mu plasmid DNA sequences is digested, that sequence will comprise the region of homology with the predetermined binding sequence in the host genome. The resulting digested CDC is referred to as a “single-stranded CDC” (ssCDC).

Alternatively, the region of DNA that shares identity with a complementary strand of a predetermined binding sequence within a host organism's genome may be attached to an active CDC obtained from any mini-Mu plasmid. In this manner, a single-stranded CDC having at least one single-stranded DNA sequence comprising a region of complementarity with the predetermined binding sequence may be constructed by ligation of such a strand into one or both of the linearized double-stranded non-Mu plasmid DNA sequences that flank the tetrameric core of the active CDC. Thus, for example, a previously obtained single-stranded DNA having the desired region of complementarity may be ligated into the CDC adjacent to one or both of the 3′—OH nucleophilic groups of the tetrameric core. Methods for ligating DNA sequences into target plasmid DNA sequences are well known in the art. See, for example, Sambrook et al. (1989) Molecular Cloning, A Laboratory Manual (2d ed.; Cold Spring Harbor Laboratory Press, Plainview, N.Y.), herein incorporated by reference. The resulting modified CDC is also encompassed by the term “single-stranded CDC” as used herein.

Where single-stranded CDCs serve as the integration vector, incubation with proteins or chemicals may enhance the effectiveness of the targeting mechanism and thereby comprise part of the targeting mechanism. For example, a single-stranded CDC may be incubated with a RecA-like protein, or biologically active fragment or variant thereof, to obtain one type of novel targeted CDC integration vector of the present invention. By “RecA-like protein” is intended any member of a family of RecA-like proteins that have the ability to recognize and promote pairing of DNA structures on the basis of shared homology. More particularly, RecA-like proteins bind to single-stranded DNA sequences, thereby forming a RecA-coated-single-stranded DNA complex that efficiently seeks out and subsequently binds to nucleotide sequences having homology to the initial single-stranded DNA sequence. Proteins like RecA recognize and promote pairing of DNA structures on the basis of shared homology, as has been shown by several in vitro experiments (Hsieh and Camerini-Otero (1989) J. Biol. Chem. 264:5089; Howard-Flanders et al. (1984) Nature 309:215; Stasiak et al. (1984) Cold Spring Harbor Symp. Quant. Biol. 49:561; Register et al. (1987) J. Biol. Chem. 262:12812). Several investigators have used RecA protein in vitro to promote formation of homologously paired triplex DNA (Cheng et al. (1988) J. Biol. Chem. 263:15110; Ferrin and Camerini-Otero (1991) Science 354:1494; Ramdas et al. (1989) J. Biol. Chem. 264:17395; Strobel et al. (1991) Science 254:1639; Rigas et al. (1986) Proc. Natl. Acad. Sci. (USA) 83:9591; and Camerini-Otero et al. U.S. Pat. Nos. 5,460,941 and 5,731,411; herein incorporated by reference).

The best characterized RecA protein is from E. coli. In addition to the native protein, a number of variant RecA-like proteins have been identified (e.g., RecA803). Further, many organisms have RecA-like strand-transfer proteins (e.g., Fugisawa et al. (1985) Nucleic Acids Res. 13:7473; Hsieh et al. (1986) Cell 44:885; Hsieh et al. (1989) J. Biol. Chem. 264:5089; Fishel et al. (1988) Proc. Natl. Acad. Sci. USA 85:3683; Cassuto et al. (1987) Mol. Gen. Genet. 208:10; Ganea et al. (1987) Mol. Cell Biol. 7:3124; Moore et al. (1990) J. Biol. Chem. 19:11108; Keene et al. (1984) Nucleic Acids Res. 12:3057; Kimiec (1984) Cold Spring Harbor Symp. 48:675; Kimiec (1986) Cell 44:545; Kolodner et al. (1987) Proc. Natl. Acad. Sci. USA 84:5560; Sugino et al. (1985) Proc. Natl. Acad. Sci. USA 85:3683; Halbrook et al. (1989) J. Biol. Chem. 264:21403; Eisen et al. (1988) Proc. Natl. Acad. Sci. USA 85:7481; McCarthy et al. (1988) Proc. Natl. Acad. Sci. USA 85:5854; Lowenhaupt et al. (1989) J. Biol. Chem. 264:20568; herein incorporated by reference.

Examples of RecA-like proteins include, but are not limited to, RecA, RecA803, and other RecA variants (Roca (1990) Crit. Rev. Biochem. Molec. Biol. 25:415), including RecA peptides (U.S. Pat. No. 5,732,411); sep1 (Kolodner et al. (1987) Proc. Natl. Acad. Sci. USA 84:5560; Tishkoffet al. Molec. Cell. Biol. 11:2593), RuvC (Dunderdale et al. (1991) Nature 354:506), DST2, KEM1, XRN1 (Dykstra et al. (1991) Molec. Cell. Biol. 11:2583), STP/DST1 (Clark et al. (1991) Molec. Cell. Biol. 11: 2576), HPP-1 (Moore et al. (1991) Proc. Natl. Acad. Sci. USA 88:9067), and uvsX. The art teaches several examples of proteins from Drosophila, plant, human, and non-human mammalian cells having biological properties similar to RecA. Such proteins are encompassed by the term RecA-like proteins.

Preferably the RecA-like protein is RecA. The purified RecA protein is a single polypeptide ranging in weight from about 37,000 to about 42,000. Although there is some variation in the sequences between bacterial strains, the RecA proteins from a variety of bacteria in general have been isolated and characterized by interspecies complementation and assays utilizing comparisons with isolated, characterized proteins. For example, cloning and characterization of RecA genes and RecA proteins from Proteus vulgaris, Erwinia carotovoria, Shigella flexneria and Escherichia coli are described by Keener et al. (1984) J. Bacteriol. 160(1):153-160. The RecA proteins produced by these organisms were demonstrated to be highly conserved among the species. In fact, the protein produced by one species could be introduced into another species where it complemented repair and regulatory defects of RecA mutations. Other bacterial RecA genes and gene products have been described by Miles et al. (1996) Mol. Gen. Genet. 204:161-165 (Agrobacterium tumefaciens C58); Goldberg et al. (1986) J. Bacteriol. 165(3):715-722 (Vibrio cholera); Better et al. (1983) J. Bacteriol. 155(1):311-316; (Rhizobium meliloti); Kokjohn et al. (1985) J. Bacteriol. 163(2):568-572; (Pseudomonas aeruginosa); and Lovett, Jr. et al. (1985) J. Biol. Chem. 260(6):3305-3313 (Bacillus subtilis). These articles detail the isolation and characterization of gene libraries and the proteins encoded by the RecA genes using techniques known to those skilled in the art including construction of gene libraries, identification of homologous genes using hybridization to probes from other more well-characterized species such as E. coli, isolation and characterization of RecA proteins using antisera to RecA proteins from E. coli, and interspecies complementation of deficient strains of E. coli using gene segments from the libraries. The isolated proteins were useful for in vitro complementation studies. RecA deficient strains and RecA clones are available from many of the laboratories cited in the above articles and from the E. coli Genetic Stock Center at Yale University.

RecA protein is typically obtained from bacterial strains that overproduce the protein. Thus, RecA may be purified from E. coli strains, such as E. coli strains JC12772 and JC15369 (available from A. J. Clark and M. Madiraju, University of California-Berkeley). These strains contain the RecA coding sequences on a “runaway” replicating plasmid vector present at a high copy numbers per cell. The recA803 protein is a highactivity variant of native RecA. Alternatively, RecA protein can also be purchased from, for example, Pharmacia (Piscataway, N.J.).

When incubated with single-stranded DNA segments, RecA protein attaches to the nucleotides, forming a nucleoprotein filament. In this nucleoprotein filament, one monomer of RecA protein is bound to about 3 nucleotides. This property of RecA to coat single-stranded DNA is essentially sequence independent, although particular sequences favor initial loading of RecA onto a polynucleotide (e.g., nucleation sequences). The nucleoprotein filament(s) can be formed on essentially any single-stranded DNA molecule.

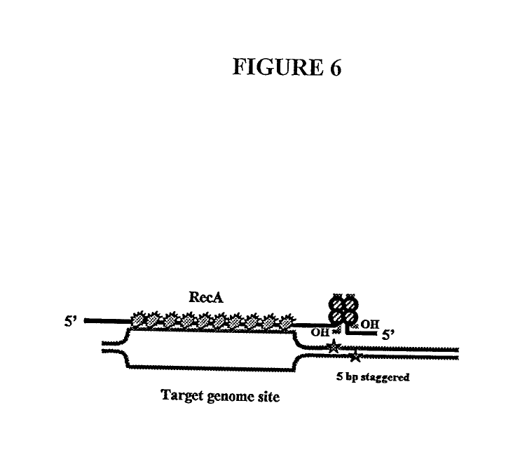

Any method known in the art for coating of single-stranded DNA sequences with a RecA-like protein may be used to obtain the RecA coated single-stranded CDC of the invention. See particularly the procedures described by Konforti and Davis (1991) J. Biol. Chem. 266(16):10112-10121); and U.S. Pat. Nos. 4,888,274; 5,460,941; 5,731,411; herein incorporated by reference. Complete coating of the complementary single-stranded non-Mu plasmid DNA sequences prevents their repair. The resulting RecA-coated single-stranded CDCs (ssCDCs) represent novel integration vectors of the invention (see FIG. 6, where the RecA-coated ssCDC is shown annealed to the predetermined target site adjacent to the target site for insertion of the Mu transposable cassette within the host genome). As used herein, the term “ssCDC” generally refers to any CDC attached to at least one single-stranded DNA navigator element.

The coating of single-stranded DNA, in this case the single-stranded CDC, with a RecA-like protein can be evaluated in a number of ways. First, protein binding to DNA can be examined using band-shift gel assays (McEntee et al. (1981) J. Biol. Chem. 256:8835). Labeled ssCDCs can be coated with RecA-like protein in the presence of ATPγ-S³⁵ and the products of the coating reactions may be separated by agarose gel electrophoresis. Following incubation of RecA-like protein with ssCDC, the RecA-like protein effectively coats single-stranded non-Mu plasmid DNA sequences flanking the MuA tetrameric core of the CDC. As the ratio of RecA-like protein monomers to nucleotides in a single-stranded region of the ssCDC increases, the ssCDC's electrophoretic mobility decreases, i.e., is retarded, due to binding of the RecA-like protein to the ssCDC. Retardation of the coated ssCDC's mobility reflects the saturation of ssCDC with RecA-like protein. An excess of RecA-like monomers to DNA nucleotides is required for efficient coating of short single-stranded DNAs. See, for example, Leahy et al. (1986) J. Biol. Chem. 261:6954.

A second method for evaluating protein binding to DNA is the use of nitrocellulose filter binding assays (Leahy et al. (1986) J. Biol. Chem. 261:6954; Woodbury et al. (1983) Biochemistry 22(20):4730-4737). The nitrocellulose filter binding method is particularly useful in determining the dissociation-rates for protein:DNA complexes using labeled DNA. In the filter binding assay, protein:DNA complexes are retained on a filter while free DNA passes through the filter. This assay method is more quantitative for dissociation-rate determinations because the separation of protein:DNA complexes from free single-stranded DNAs is very rapid.

Thus, the novel integration vectors of the invention comprise an active CDC and a navigator element capable of localizing the CDC to a predetermined binding site in the genome of an organism of interest. Where the localization function or targeting function of the navigator element depends not only on the binding of the navigator element to the host genomic DNA itself but also depends on the presence or status (e.g., conformation, activity) other molecules or chemical entities, the navigator element is said to be part of a “targeting mechanism.” The novel targeted CDC integration vectors of the invention comprise any type of “navigator element,” for example, a chemical entity, a DNA molecule, an RNA molecule, a synthetic analog of RNA or DNA, a peptide nucleic acid (PNA), a protein, or any combination thereof. By “peptide nucleic acid” is intended a nucleic acid mimic, e.g., DNA mimic, in which the deoxyribose phosphate backbone is replaced by a pseudopeptide backbone and only the four natural nucleobases are retained (see, for example, Hyrup et al. (1996) Bioorganic Med. Chem. 4:5). The neutral backbone of PNAs has been shown to allow for specific hybridization to DNA and RNA under conditions of low ionic strength. The synthesis of PNA oligomers can be performed using standard solid-phase peptide synthesis protocols as described in, for example, Hyrup et al. (1996) Bioorganic Med. Chem. 4:5, and Perry-O'Keefe et al. (1996) Proc. Natl. Acad. Sci. USA 93:14670. Such PNA sequences can be used, for example, as antisense agents for sequence-specific modulation of gene expression. Thus, the localization or targeting function of the navigator element may be accomplished by means of a protein that is bound to the active CDC by interaction with a non-Mu plasmid domain having a binding site for the protein. This protein may then bind to another protein, which in turn binds to a predetermined binding sequence, such as further described herein. In such an embodiment, the proteins may be native proteins or they may be engineered or otherwise modified to comprise one or more components useful in the practice of the present invention. For example, the proteins may be modified to bind to each other by the addition of binding regions, as described in, for example, Fields and Song (1989) Nature 340:245-246; see also, Vidal and Endoh (1999) Trends in Biotechnol. 17:374-381. Additionally, the targeting mechanism may comprise a wellknown set of reagents and/or compounds known to bind one another, for example, biotin and streptavidin (see generally, Antibodies: A Laboratory Manual, Harlow and Lane, eds., Cold Spring Harbor Laboratory (1988)). One of skill in the art will appreciate that the targeting mechanism may be provided by any combination of elements providing suitable localization of the active CDC; for example, any combination of DNA, RNA, synthetic analogs of DNA or RNA, proteins, or chemical. See, for example, Zhang et al. (2000) Methods Enzymol. 318:399-419. A chemical navigator or targeting mechanism might involve, for example, a steroid bound to the active CDC which would bind a steroid receptor, thereby targeting integration to a site adjacent to the steroid receptor.

Various navigator elements may be used in the compositions and methods of the invention. Navigator elements may be composed of DNA, PNA, RNA, protein, or other chemical entity or any combination thereof so long as the navigator element is capable of providing localization to a predetermined binding site in the genome.

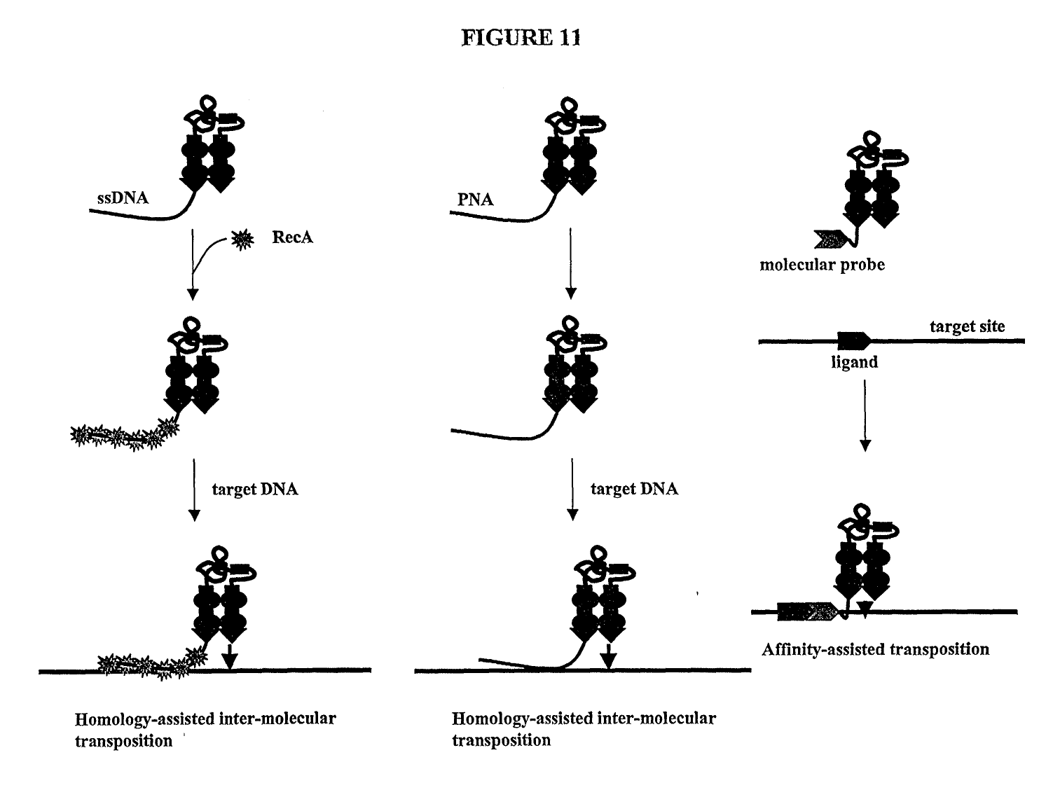

In some embodiments of the invention, the navigator element which facilitates localization or targeting of the vector is a peptide nucleic acid, or PNA. Peptide nucleic acids are able to bind complementary DNA tracts with high affinity and selectivity and thus mimic the behavior of oligonucleotides (depicted in FIG. 11). However, PNA is more stable in cells than single-stranded DNA (ssDNA). In another embodiment of the invention, the navigator element is a protein which has a DNA binding function and is capable of binding to DNA at one or more specific DNA sites. In this way, the targeting or localization function of the navigator element is provided by a protein-DNA interaction. For example, a protein used to create a navigator element may be a trans-acting factor which binds to the promoter or regulatory region of a single gene or to the promoter or regulatory region of a group of genes.

In some embodiments, the localization or targeting function of the navigator element is provided by a protein-protein interaction. For example, in such an embodiment, a protein navigator element of the CDC is capable of binding to another protein, such as a cellular trans-acting factor, which in turn is bound to DNA, and in this way the protein-protein interaction contributes to the specific targeting of the CDC (for example, see diagram of “affinity-assisted transposition” in FIG. 11).

In some embodiments, the localization or targeting function may be provided by a chemical-protein interaction. For example, in such an embodiment, a steroid navigator element may be attached to the CDC; the steroid then binds to a steroid receptor, which in turn will bind to the steroid/ steroid receptor complex binding domain in the genome and the CDC complex will integrate into the genome nearby.

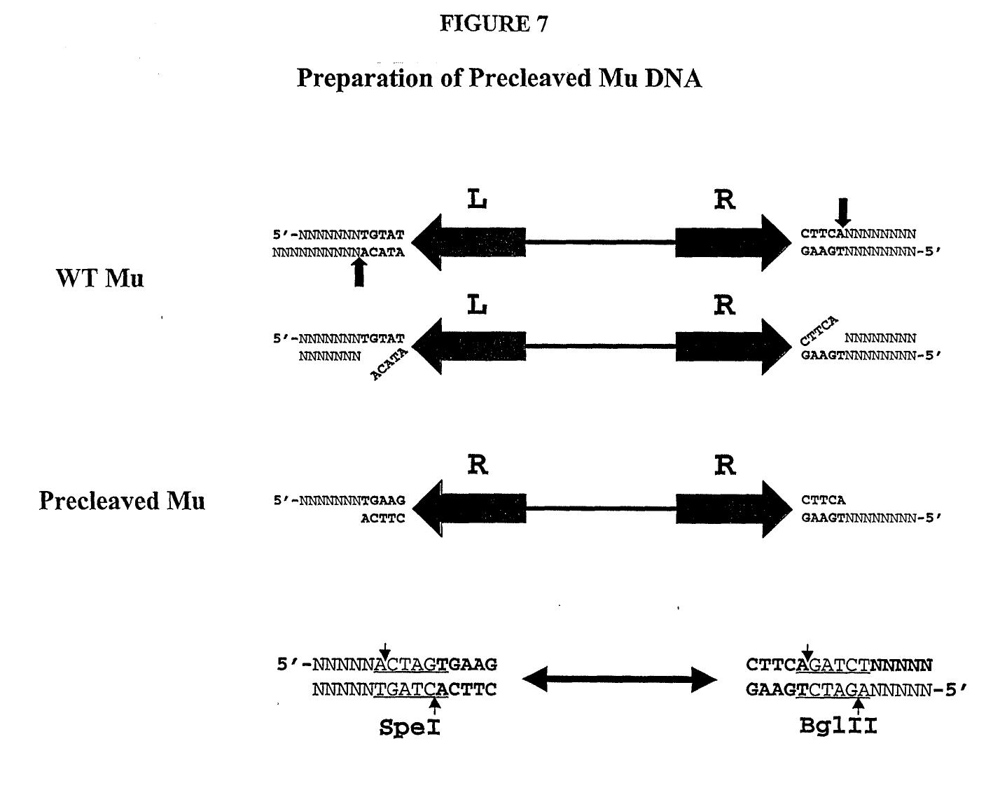

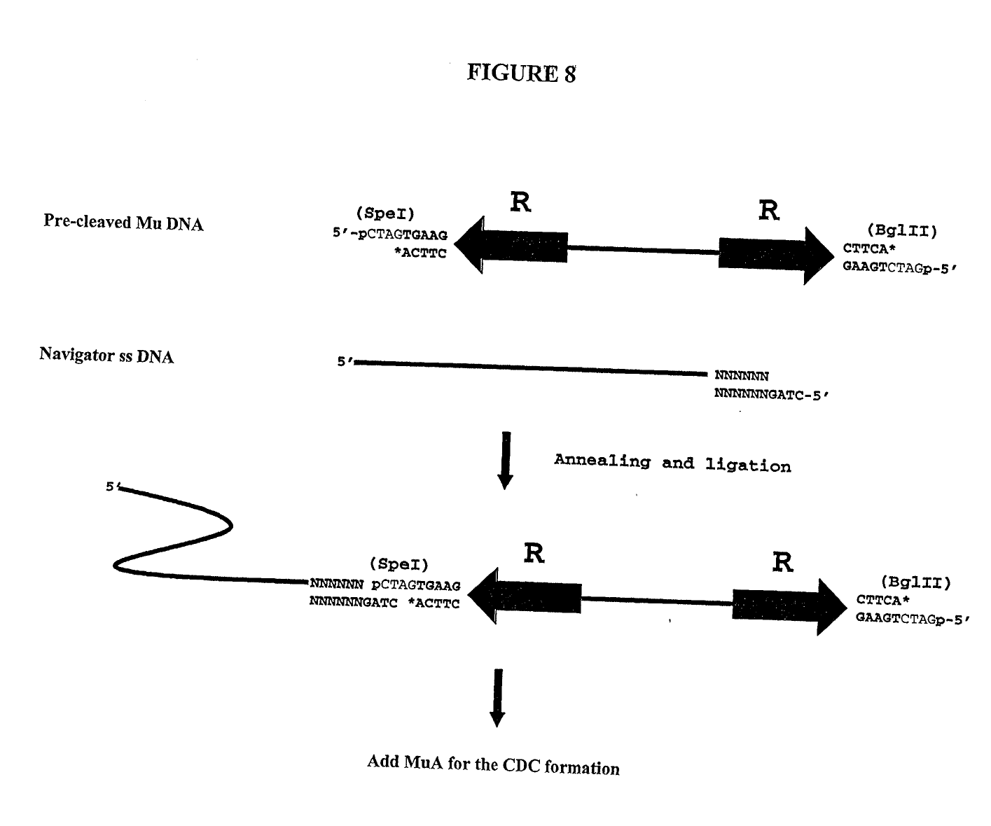

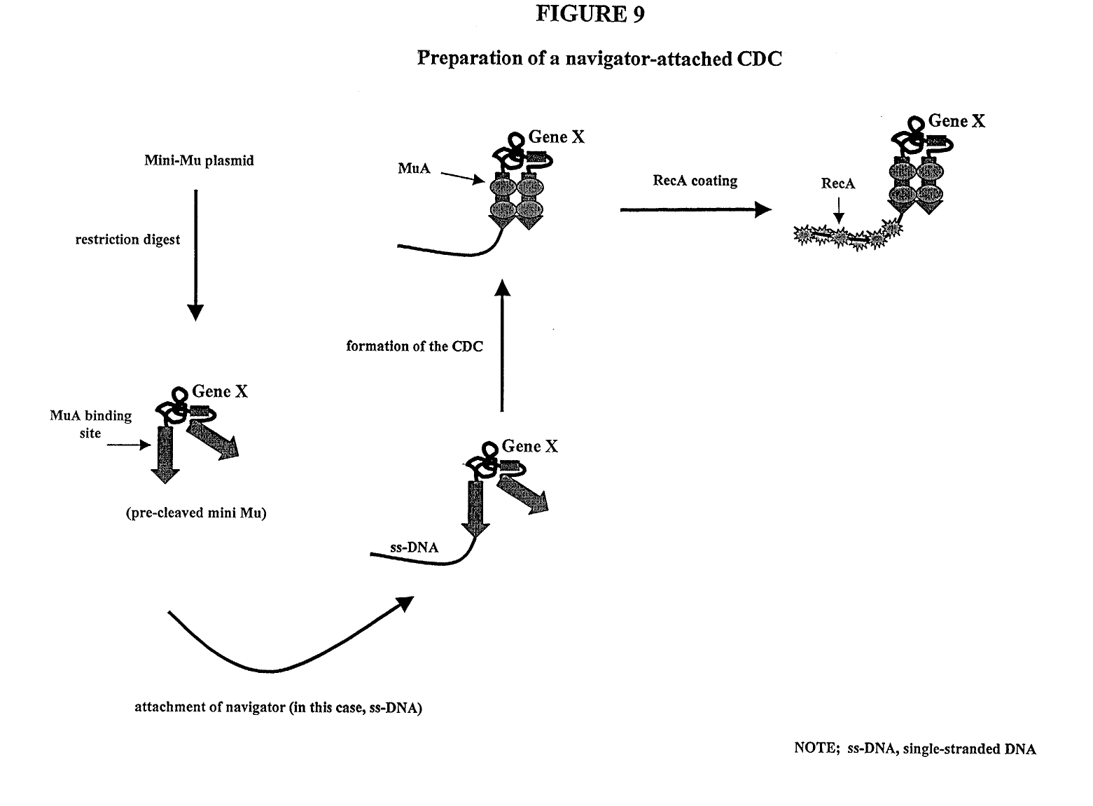

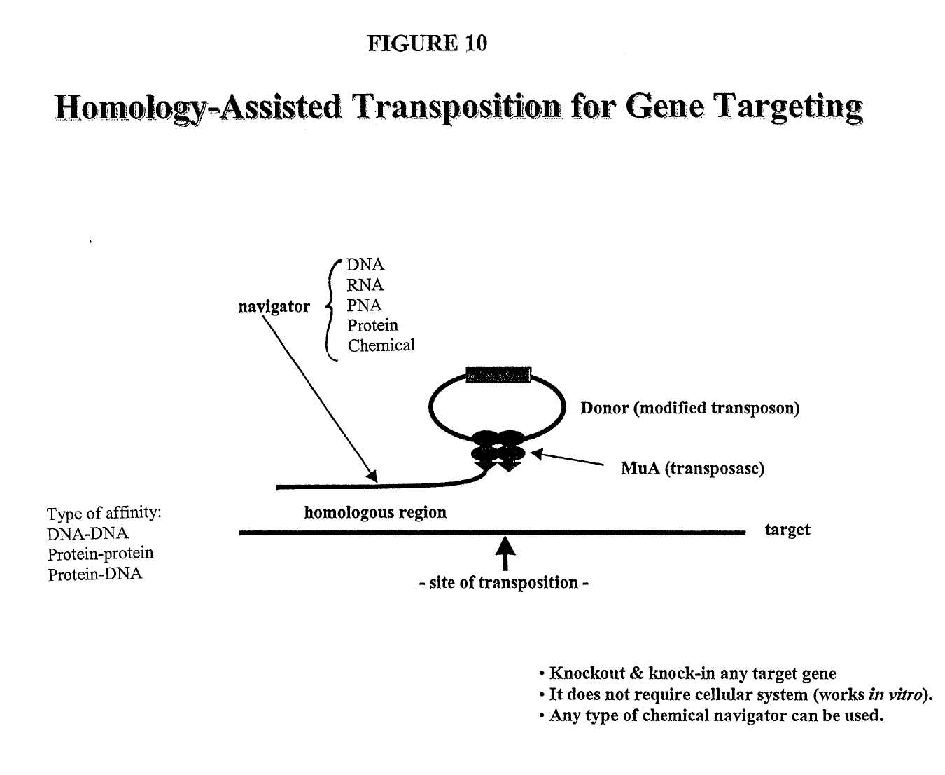

In any embodiment where a navigator element is used, the navigator element can be, for example, a chemical entity, an RNA molecule, a synthetic analog of RNA of DNA, a peptide nucleic acid (PNA), or a protein, as previously described, or any combination thereof. A navigator element serves to direct the integration of the Mu transposable cassette into a predetermined target site adjacent to a predetermined binding site within the host genome or to enhance the efficiency of localization or integration (see FIGS. 10 and 11). By predetermined binding site is intended that a navigator tends to localize to a particular sequence within the host genome; such localization may be accomplished by nucleic acid interaction, by interaction between proteins, by interaction between protein and nucleic acid analogs, or by any other interaction involving the navigator element whereby the navigator element is localized to a particular sequence within the host genome. By predetermined target site is intended a location in the host genome into which insertion of the Mu transposable cassette is desirable and may be achieved by localization of the navigator element to the predetermined binding site. Thus, in one embodiment, the navigator element is a single-stranded DNA sequence designed to comprise a region of homology with a predetermined binding sequence in the host genome that resides adjacent to the target site for insertion of the Mu transposable cassette. Where a navigator element is single-stranded DNA, the navigator may be attached to the precleaved mini-Mu plasmid using standard annealing and ligation techniques. When serving as the navigator element of an active CDC, the attached single-stranded DNA can subsequently be incubated with a RecA-like protein, as previously described, to further facilitate binding of the single-stranded DNA with the region of homology in the host DNA (see FIGS. 9 and 11). Such navigator-assisted transposition is referred to herein as homology-assisted intermolecular transposition. This type of transposition is also involved in another embodiment of the invention where the navigator element is a PNA sequence that hybridizes to a predetermined binding site adjacent to the predetermined target site for integration of the Mu transposable cassette. (see FIG. 11). In one embodiment a navigator element is a molecular probe that binds to a ligand in the target nucleotide sequence that is positioned adjacent to the predetermined target site for integration of the Mu transposable cassette (see FIG. 11). Such navigator-assisted transposition is referred to herein as affinity-assisted transposition. In this embodiment, the predetermined binding site is the target nucleotide sequence to which the ligand binds. In some embodiments of the present invention, the novel integration vectors are navigator-attached CDCs that are obtained from precleaved or “precut” mini-Mu plasmids. By “precleaved” or “precut” mini-Mu plasmid is intended a wild-type or derivative mini-Mu plasmid that has been subjected to restriction enzyme digestion to cleave the double-stranded non-Mu plasmid DNA domain within at least one region, thereby linearizing this domain prior to formation of the active CDC. Preferably a derivative mini-Mu plasmid is used, for example a derivative mini-Mu plasmid that comprises two Mu right end recognition sequences in inverted orientation. Strand transfer is most efficient when a pair of Mu right end recognition sequences is used with precleaved mini-Mu plasmids. See, for example, Craigie and Mizuuchi (1987) Cell 51:493-501, and Namgoong et al. (1994) J. Mol. Biol. 238:514-527. Each of these Mu right end recognition sequences can be the complete Mu right end recognition sequence, i.e., having all three MuA transposase binding sites (i.e., attR1, attR2, and attR3) in natural orientation and order, or can comprise just one or more of the binding sites, for example, the attR1 and attR2 sites (see Savilahti et al. (1995) EMBO J. 14:4893-4903). As with other mini-Mu plasmids, the Mu end recognition sequences flank an internal nucleotide sequence, which can further comprise, for example, the Mu transpositional enhancer sequence, a scorable marker gene, and/or a sequence of interest to be targeted into the host genome. Preferably the restriction enzyme is chosen such that cleavage takes place within a region of nucleotides adjacent to a Mu end recognition sequence of the Mu transposable cassette, thereby generating a 5′-overhang sequence that immediately flanks the DNA cleavage site just outside this Mu end recognition sequence. Digestion with two restriction enzymes specific for restriction site sequences within the non-Mu plasmid DNA flanking the Mu transposable cassette can generate a precleaved mini-Mu plasmid comprising a Mu transposable cassette immediately flanked by 5′-overhang sequences (see, for example, FIGS. 7 and 8, where SpeI and BglII are used in the restriction digest). For preparation and use of precleaved mini-Mu plasmids (also referred to as precleaved Mu DNA), see Craigie and Mizuuchi (1987) Cell 51:493-501; Mizuuchi and Mizuuchi (1989) Cell 58:399-408; Savilahti et al. (1995) EMBO J. 14:4893-4903; Haapa et al. (1999) Nucleic Acids Res. 27:2777-2784; and Haapa et al. (1999) Genome Res. 9:308-315. In some embodiments of the invention, a navigator element may be attached or bound to the precleaved mini-Mu plasmid, using standard enzymatic or chemical procedures known in the art, to obtain a navigator-attached precleaved mini-Mu plasmid (see FIG. 9).

Following attachment of the navigator element, the precleaved mini-Mu plasmid may be subjected to an in vitro reaction as previously described to add MuA and/or other proteins such as RecA to the CDC in order to obtain an active CDC with enhanced targeting ability (see FIG. 11). In vitro requirements for formation of an active CDC from a precleaved mini-Mu plasmid are relaxed (see, for example, Craigie and Mizuuchi (1987) Cell 51:493-501, and Mizuuchi and Mizuuchi (1989) Cell 58:399-408). Thus, active CDC formation can take place in the absence of superhelicity of the mini-Mu plasmid and the E. coli HU protein. The elimination of these requirements is beneficial, as HU protein is not readily commercially available, and isolation of plasmids with high superhelicity is more time consuming and labor intensive. Further, several enzymatic treatments following CDC formation, such as restriction digest, ExoIII digest, ligation, and attachment of other navigators or binding of substances which enhance localization can be minimized with the use of the precleaved mini-Mu plasmid. Thus, use of the precleaved mini-Mu plasmids can minimize manipulation, and potentially prolonged incubation, of the CDC in different environments.

Thus, the novel integration vectors of the invention may be obtained using mini-MU plasmids and any other necessary or helpful proteins, such as, for example, MuA transposase, the bacterial proteins HU, IHF, and a RecA-like protein, or biologically active variants or fragments thereof. Such proteins may be produced in vivo by the host genome, for example as the result of previous genetic engineering of the genome, or the proteins may be introduced along with the integration vectors during or after transformation of the host genome with the integration vectors. Such introduction may be direct or indirect (for example, by cotransformation of an integration vector with another DNA sequence encoding MuA transposase). Thus, active CDCs may be formed within the host cell where the appropriate elements and sequences exist within the cell.

Where purified proteins are to be used, methods for obtaining these purified native proteins or biologically active variants or fragments thereof are known in the art. See, for example, Craigie and Mizuuchi (1985) J. Biol. Chem. 260:1832-1835 (cloning of the MuA gene and purification of MuA); Craigie et al. (1985) Proc. Natl. Acad. Sci. USA 82:7570-7574, Rouviere-Yaniv and Gros (1975) Proc. Natl. Acad. Sci. USA 72:3428-3432, Dixon and Komberg (1984) Proc. Natl. Acad. Sci. USA 81:424-428, and Surette et al. Cell 49:253:226 (purification of HU); Wu and Chaconas (1994) J. Biol. Chem. 269:28829-28833, and the references cited therein (MuA, HU, and IHF); Yang et al. (1995) EMBO J 14:2374-2384 (native MuA and variants thereof, and HU); and Shibita et al. (1982) J. Biol. Chem. 257:370, Shibita et al. (1983) Methods Enzymol. 100:197, Cox et al. (1981) J. Biol. Chem. 256(9):4676, and Cox et al. (1981) Proc. Natl. Acad. Sci. USA 78:3433 (purified RecA); herein incorporated by reference.

By “purified” is intended the protein, or biologically active variant or fragment thereof, is substantially or essentially free from components that normally accompany or interact with the protein as found in its naturally occurring environment. Thus a purified protein is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, (by dry weight) of contaminating protein. Thus, when the MuA, HU, IHF, or RecA-like protein or biologically active variant or fragment thereof is recombinantly produced, culture medium represents less than about 30%, 20%, 10%, or 5% (by dry weight) of chemical precursors or non-protein-of-interest chemicals.

By “fragment” is intended a portion of the amino acid sequence and hence protein encoded thereby. For example, a biologically active portion of the MuA, HU, IHF, or RecA-like protein can be prepared by isolating a portion of their respective coding sequences, expressing the encoded portion of the respective protein (e.g., by recombinant expression in vitro), and assessing the activity of the encoded portion of the respective protein. The coding sequences for these proteins are known in the art. See, for example, Grimaud (1996) Virology 217(1):200-210 for the nucleotide sequence for the Mu bacteriophage (GenBank Accession No. AF083977), which identifies the coding sequence for the MuA transposase (GenBank Accession No. AAF01083); Miller (1984) Cold Spring Harb. Symp. Quant. Biol. 49:691-698 for the coding sequence for the IHF alpha-subunit (GenBank Accession No. P06984) and Flamm and Weisberg (1985) J. Mol. Biol. 183(2): 117-128 for the coding sequence for the IHF beta-subunit (GenBank Accession No. P08756); GenBank Accession No. U82664, nucleotides 40901-41173, which code for the HU protein (GenBank Accession No. AAB40196); and Keener et al. (1984) J. Bacteriol. 160(1):153-160 and the references cited elsewhere herein for coding sequences for RecA-like proteins.

By “variant” MuA, HU, IHF, or RecA-like protein is intended a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Variant MuA transposase, HU, IHF, and RecA-like proteins useful in carrying out the construction of the integration vectors of the invention are biologically active, that is they continue to possess the desired biological activity of the native protein. Thus, a variant MuA transposase effectively binds to the binding sites of the native or mutant CDC and assists with intermolecular transposition of the Mu transposable cassette from the integration vector of the invention into a predetermined target site within the genome of a host organism. Similarly, a variant HU protein interacts with a mini-Mu plasmid donor near an attL1 binding site within the Mu transposable cassette to facilitate the role of this binding site in CDC assembly. A variant IHF protein, when present in the reaction mixture, binds to its specific site in the Mu transpositional enhancer sequence to achieve the optimal geometrical conformation of the mini-Mu plasmid domain comprising the Mu transposable cassette. A variant RecA-like protein binds to single-stranded DNA, more particularly the single-stranded non-Mu plasmid DNA sequences flanking the MuA tetrameric core of the cleaved donor complex, and facilitates binding of the sequence(s) sharing homology to the predetermined binding site adjacent to the predetermined target sequence within the host organism's genome. Such variant proteins may result from, for example, genetic polymorphism or from human manipulation.

The known amino acid sequences for the native MuA transposase, HU, IHF, RecA-like, and other proteins may be altered in various ways including amino acid substitutions, deletions, truncations, and insertions. Methods for such manipulations are generally known in the art. For example, amino acid sequence variants of these proteins can be prepared by mutations in their respective coding sequences. Methods for mutagenesis and nucleotide sequence alterations are also well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and references cited therein. Guidance as to appropriate amino acid substitutions that do not affect biological activity of the protein of interest may be found in the model of Dayhoff et al. (1978) Atlas of protein Sequence and Structure (Natl. Biomed. Res. Found., Washington, D.C.), herein incorporated by reference. Conservative substitutions, such as exchanging one amino acid with another having similar properties, may be preferred.

Thus, the MuA transposase, HU, IHF, RecA-like proteins, and other proteins used to obtain the integration vectors of the invention include both the native (i.e., naturally occurring) proteins as well as biologically active variants. Obviously, where mutations are made in their respective DNA coding sequences to obtain variant forms, the mutations must not place the sequence out of reading frame and preferably will not create complementary regions that could produce secondary mRNA structure. See, for example, EP Patent Application Publication No. 75,444.

The deletions, insertions, and substitutions of the amino acid sequences for these proteins are not expected to produce radical changes in the characteristics of the respective proteins. However, when it is difficult to predict the exact effect of the substitution, deletion, or insertion in advance of doing so, one skilled in the art will appreciate that the effect will be evaluated by routine screening assays. Thus, activity of variant MuA transposase, HU, and IHF proteins can be evaluated using the standard in vitro transposition reaction (Mizuuchi et al. (1992) Cell 70:303-311), while activity of a variant RecA-like protein can be evaluated using band-shift gel assays (McEntee et al. (1981) J. Biol. Chem. 256:8835) or nitrocellulose filter binding assays (Leahy et al. (1986) J. Biol. Chem. 261:6954; Woodbury et al. (1983) Biochemistry 22(20):4730-4737); herein incorporated by reference.

Where standard in vitro reaction conditions are to be used for producing active CDCs, variant MuA transposase proteins preferably retain amino acid residues 1-76 of the native MuA protein (the so-called N-terminal domain). However, altered reaction conditions are used, the requirements for active CDC formation may be relaxed. For example, when DMSO is included in the standard reaction conditions, active CDCs can be obtained using variant MuA transposase proteins lacking the N-terminal domain. See Mizuuchi and Mizuuchi (1989), Cell 58:399-408.

Biologically active variants of a native MuA transposase, HU, IHF, RecA-like , or other protein will have at least about 40%, 50%, 60%, 65%, 70%, generally at least about 75%, 80%, 85%, preferably at least about 90%, 91%, 92%, 93%, 94%, 95%, 96%, 97%, and more preferably at least about 98%, 99% or 100% sequence identity to the amino acid sequence for the native protein as determined by sequence alignment programs described below using default parameters. A biologically active variant of these proteins may differ from the native protein by as few as 1-15 amino acid residues, as few as 1-10, such as 6-10, as few as 5, as few as 4, 3, 2, or even 1 amino acid residue.

The following terms are used to describe the sequence relationships between two or more polypeptides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, a segment of a full-length amino acid sequence, or the complete amino acid sequence.

(b) As used herein, “comparison window” makes reference to a contiguous and specified segment of an amino acid sequence, wherein the amino acid sequence in the comparison window may comprise additions or deletions (i.e., gaps) compared to the reference sequence (which does not comprise additions or deletions) for optimal alignment of the two sequences. Generally, the comparison window is at least 20 contiguous amino acids in length, and optionally can be 30, 40, 50, 100, or longer. Those of skill in the art understand that to avoid a high similarity to a reference sequence due to inclusion of gaps in the amino acid sequence a gap penalty is typically introduced and is subtracted from the number of matches.

Methods of alignment of nucleotide and amino acid sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al. (1990) J. Mol. Biol 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a MuA transposase, HU, or IHF protein of the invention. BLAST protein searches can be performed with the BLASTX program, score=50, wordlength=3, to obtain amino acid sequences homologous to these proteins. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See http://www.ncbi.nlm.nih.gov. Alignment may also be performed manually by inspection.

Unless otherwise stated, sequence identity/similarity values provided herein refer to the value obtained using GAP version 10 using the following parameters: % identity using GAP Weight of 50 and Length Weight of 3; % similarity using Gap Weight of 12 and Length Weight of 4, or any equivalent program. By “equivalent program” is intended any sequence comparison program that, for any two sequences in question, generates an alignment having identical nucleotide or amino acid residue matches and an identical percent sequence identity when compared to the corresponding alignment generated by GAP Version 10.

GAP uses the algorithm of Needleman and Wunsch (1970) J. Mol. Biol. 48: 443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the amino acid sequence in the comparison window may comprise additions or deletions (i.e., gaps) as compared to the reference sequence (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the window of comparison, and multiplying the result by 100 to yield the percentage of sequence identity.

(e) The term “substantial identity” in the context of a peptide indicates that a peptide comprises an amino acid sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman and Wunsch (1970) J. Mol. Biol. 48:443-453. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

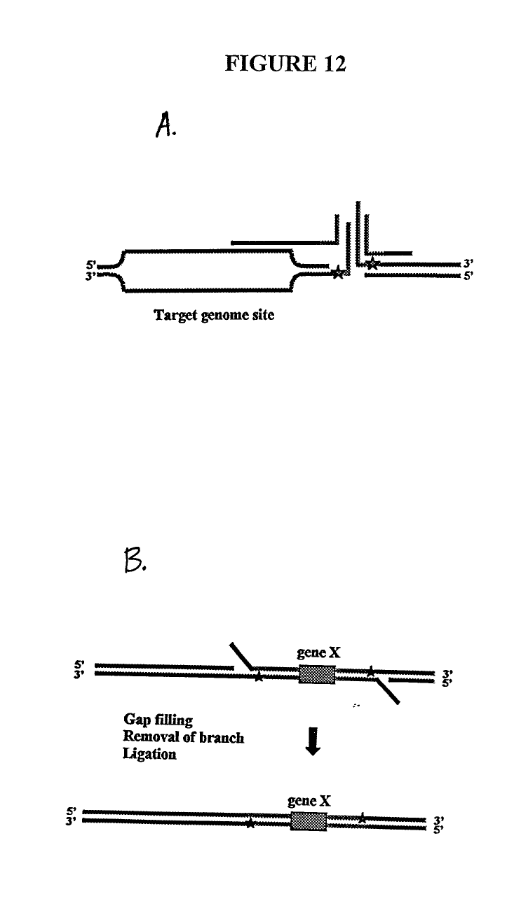

The novel integration vectors of the invention are useful in methods directed to targeted genetic manipulation of a host organism's genome. For example, when a host cell is transformed with a RecA-coated single-stranded CDC of the invention, the flanking RecA-coated single-stranded DNA navigator comprising the region of homology hybridizes to the predetermined binding sequence adjacent to the predetermined target site within the host organism's genome. Following this RecA-assisted hybridization, the MuA protein bound as part of the single-stranded CDC facilitates subsequent strand transfer between the CDC and the predetermined target site, leading to insertion of the entire Mu transposable cassette into the target site of the organism's genome (see FIG. 12). Depending upon the location of the predetermined target site, insertion of the Mu transposable cassette can be used to alter gene expression within a host organism, such as knocking out expression of an endogenous gene, creating expression of a transgene, enhancing expression of a desired gene product or simultaneously knocking out expression of an endogenous gene and promoting or creating expression of a nucleotide sequence that encodes a variant of the disrupted endogenous gene product.

The mini-Mu plasmid serving as the starting point for formation of an integration vector of the invention may be constructed such that the internal DNA sequence of the Mu transposable cassette further comprises a scorable marker gene to facilitate selection of transformed host cells comprising the Mu transposable cassette inserted within the predetermined target site. See, for example, Chaconas et al. (1981) Gene 13:3746. Scorable marker genes include, for example, selectable marker genes and assayable reporter genes.

Selectable marker genes confer resistance to a particular selection agent, and thus allow for selection of transformed cells/tissues in the presence of such a selection agent. Selectable marker genes include, but are not limited to, genes encoding antibiotic resistance, such as those encoding neomycin phosphotransferase II (NEO) (Fraley et al. (1986) CRC Critical Review in Plant Science 4:1-25) and hygromycin phosphotransferase (HPT or HYG) (Vanden Elzen et al. (1985) Plant Mol. Biol. 5:299; Shimizu et al. (1986) Mol. Cell Biol. 6:1074, as well as genes conferring resistance to herbicidal compounds, such as glufosinate ammonium, bromoxynil, and 2,4-dichlorophenoxyacetate (2,4-D).

By “assayable reporter gene” is intended any scorable marker gene that can be assayed for its presence and/or expression. Reporter genes generally encode a protein whose activity can be assayed to determine whether the reporter gene is present and/or is being expressed. Preferably the protein can be assayed using nonlethal methods. Use of such assayable reporter genes as opposed to selectable marker genes to facilitate selection of transgenic plants is disclosed in detail in the copending application entitled “Recovery of Transformed Plants Without Selectable Markers by Nodal Culture and Enrichment of Transgenic Sectors,” U.S. patent application Ser. No. 08/857,664, filed May 16, 1997, herein incorporated by reference.

With assayable reporter genes, generally too there is some sort of chemical, biological, or physical assay available that will determine the presence or absence or change in amount of the expression product of the gene. In certain embodiments in which the assayable reporter gene produces an enzyme involved in a metabolic pathway, the assay may determine the presence or absence of, or a change in the amount of, a metabolite produced directly by the enzyme, or the presence or absence of, or a change in the amount of, a metabolite produced indirectly by the enzyme, or the presence or absence of, or a change in the amount of, the final product of the metabolic pathway, rather than the presence or absence of the expression product (the enzyme) itself. For example, such an enzyme might be involved in a metabolic pathway that produces oils having a particular fatty acid makeup. It will also be apparent to those of skill in the art that many forms of assay techniques are available to detect the presence and/or expression of reporter genes. For example, any expressed protein capable of detection by ELISA could be assayed by using the associated ELISA, or a modification in the amount of a specific fatty acid could be determined using the appropriate biochemical analytical technology (GCMS, for example); or a bioassay could be used (for example, expression of a crystal protein toxin from Bacillus thuringiensis (Bt) could be determined by screening for deleterious effects of transformed plant tissue on insects or insect larvae that are susceptible to the crystal protein toxin).

Those of skill in the art will also recognize that the presence of the assayable reporter gene can be detected directly using DNA amplification techniques known in the art, including, but not limited to PCR, RT-PCR, or LCR, for example. By way of illustration, the assayable reporter gene could be an embryo-specific gene such as a desaturase under the control of an embryo-specific promoter. Genetic modification using such a gene construct would be expected to modify seed oil profiles, without affecting expression in leaves. A properly performed PCR screen would detect the presence of the sequence in transformed plants. It will also be recognized that any gene that can be amplified using amplification technology such as PCR can serve as an assayable reporter gene in the present invention.

Reporter genes are particularly useful to quantify or visualize the spatial pattern of expression of a gene in specific tissues. Commonly used reporter genes include, but are not limited to, β-glucuronidase (GUS) (Jefferson (1987) Plant Mol. Biol. Rep. 5:387); Bgalactosidase (Teeri et al. (1989) EMBO J. 8:343-350); luciferase (Riggs et al. (1987) Nucleic Acids Res. 15(19): 8115; Luehrsen et al. (1992) Methods Enzymol. 216: 397-414); chloramphenicol acetyltransferase (CAT) (Lindsey and Jones (1987) Plant Mol. Biol. 10:43-52); green fluorescence protein (GFP) (Chalfie et al. (1994) Science 263:802); and the maize genes encoding for anthocyanin production (Ludwig et al. (1990) Science 247:449).

Other examples of assayable reporter genes include, but are not limited to, the oxalate oxidase gene, which has been isolated from wheat (Dratewka-Kos et al. (1989) J. Biol. Chem. 264:4896-4900 (the “germin” gene) and barley (WO 92/14824); the oxalate decarboxylase gene, which has been isolated from Aspergillus and Collybia (see WO 94/12622); other enzymes that utilize oxalate; other enzymes such as polyphenol oxidase, glucose oxidase, monoamine oxidase, choline oxidase, galactose oxidase, 1-aspartate oxidase, and xanthine oxidase, and the like.

As those of skill in the art will recognize, the assay for reporter genes will vary with the nature of the expression product. For example, an enzymatic assay can be used in those instances where the expression product is an enzyme, such as in the case of transformation with a gene encoding oxalate oxidase or oxalate decarboxylase. A visual or colorimetric assay would be appropriate for cells or tissues transformed with a GFP gene. As those skilled in the art will also recognize, when an enzymatic assay is appropriate, the existence of an assay in the art would be particularly useful. Furthermore, as noted above, other assay techniques (e.g., PCR for the assayable reporter itself, or ELISA, or a bioassay, or chemical analytical methods such as GCMS) will be appropriate in the performance of the various embodiments of the invention.

In a further alternative embodiment of the present invention, the assay can involve a procedure that measures a loss of, or a decrease in the level of expression of, a measurable product that is normally present or that is normally expressed at higher levels. For example, a gene disruption may decrease or eliminate gene expression from a particular gene copy, and antisense or co-suppression technology can be used to downregulate the expression of a particular gene. An appropriate assay that would detect the disappearance of or decrease in amount of the expression product or a metabolic product can be used to detect such alteration in expression.

For further information on the use of scorable marker genes, see generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) The Operon, pp. 177-220; Hu et al. (1987) Cell 48:555-566; Brown et al. (1987) Cell 49:603-612; Figge et aL (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; M. Gossen (1993) Ph.D. Thesis, University of Heidelberg; Reines et al. (1993) Proc. Natl. Acad. Sci. USA 90:1917-1921; Labow et al. (1990) Mol. Cell Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res, 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Gatz et al. (1992) Plant J. 2:397-404; A. L. Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook Exp. Pharmacol. 78; Gill et al. (1988) Nature 334:721-724. Such disclosures are herein incorporated by reference.

When present in the Mu transposable cassette, the scorable marker gene is operably linked to regulatory regions, i.e., to a promoter and terminator sequence, that drive expression of the scorable marker within an organism transformed with the novel integration vectors of the invention. Thus the scorable marker gene can be constructed as part of an expression cassette as described elsewhere herein and inserted within the internal DNA sequence of the Mu transposable cassette.

Subsequent removal or “knockout” of the scorable marker gene from stably transformed cells may be of interest, such as when the marker gene is undesirable, e.g., from an environmental point of view. Thus, in one embodiment of the invention, the scorable marker gene within the Mu transposable cassette is engineered with flanking target sequences for a site-specific recombination enzyme. These flanking target sequences may or may not be identical so long as the recombinase protein is capable of recognizing and interacting with the target sequences. The presence of these target sequences allows for the specific deletion of the marker gene from the genome of a host cell having the Mu transposable cassette integrated within its genome. This enables construction of marker-free transformed cells having the desired phenotypic change. In this embodiment of the invention, the desired outcome from transformation with an integration vector of the invention is achieved in a two-step process. In the first step, integration of the Mu transposable cassette into the genome of the host cell is accomplished by transformation and selection of host cells testing positive for the scorable marker gene. In the second step, removal of the marker gene from the host genome is accomplished by a site-specific recombinase, which interacts with the target sequences flanking the marker gene.

The site-specific recombinase can be provided in cis with the scorable marker sequence, i.e., within the Mu transposable cassette comprising the scorable marker, or in trans, i.e., on a different transformation vector. When provided in cis, the recombinase DNA sequence should be operably linked to an inducible promoter (for example, chemical-inducible promoters and temperature-inducible promoters). In this manner, expression of the recombinase protein can be controlled to take place only after targeted integration has taken place. Where the DNA sequence encoding the recombinase enzyme is provided in cis and under the control of an inducible promoter, targeted integration of the Mu transposable cassette and subsequent removal of the scorable marker gene may be accomplished using only one transformation step.

Several site-specific recombination systems are known in the art, all of which are encompassed for the intended use of removing an undesirable scorable marker gene following targeted integration of the Mu transposable cassette within the host organism's genome. For purposes of the present invention, preferably the site-specific recombination system consists of a site-specific recombinase enzyme and a target sequence for said enzyme. The recombination system of choice will depend upon the host organism. Examples of such systems are the pAMβ1 resolvase having as target sequence the pAMβ1 res sequence (Janniere et al. (1993) in Bacillus subtilis and Other Gram-positive Bacteria: Biochemistry, Physiology and Molecular Genetics, ed. Sonenshein et al. (American Society for Microbiology, Washington, D.C.), pp. 625-644); the phage P1 Cre enzyme having as target sequence the P1 lox site (Hasan et al. (1994) Gene 150:51-56); and the yeast FLP recombinase enzyme having as target sequence the FRT site (Cox (1993) Proc. Natl. Acad. Sci. USA 80:4223-4227). When the host organism is a plant, preferably the recombinase system is the FLP/FRT or Cre/lox system.

The FLP recombinase is a protein that catalyzes a site-specific reaction that is involved in amplifying the copy number of the 2μ plasmid of Saccharomyces cerevisiae during DNA replication. FLP protein has been cloned and expressed. See, for example, Cox (1993) Proc. Natl. Acad. Sci. USA 80:4223-4227, herein incorporated by reference. The FLP recombinase for use in the invention may be that derived from the genus Saccharomyces. It may be preferable to synthesize the recombinase using plant-preferred codons for optimum expression in a plant of interest. See U.S. Pat. No. 5,929,301, herein incorporated by reference. The bacteriophage recombinase Cre catalyzes site-specific recombination between two lox sites. The Cre recombinase is known in the art. See, for example, Guo et al. (1997) Nature 389:40-46; Abremski et al. (1984) J. Biol. Chem. 259:1509-1514; Chen et al. (1996) Somat. Cell Mol. Genet. 22:477-488; and Shaikh et al. (1977) J. Biol. Chem. 272:5695-5702; herein incorporated by reference. The Cre recombinase may also be synthesized using plant-preferred codons.

Recombination sites for use in the invention are known in the art and include FRT sites (see, for example, Schlake et al. (1994) Biochemistry 33:12746-12751; Huang et al. (1991) Nucleic Acids Res. 19:443-448; Sadowski (1995) Prog. Nuc. Acid Res. Mol. Bio. 51:53-91; Cox (1989) Mobile DNA, ed. Berg and Howe (American Society of Microbiology, Washington D.C.), pp. 116-670; Dixon et al. (1995) 18:449-458; Umlauf et al. (1988) EMBO J. 7:1845-1852; Buchholz et al. (1996) Nucleic Acids Res. 24:3118-3119; Kilby et al. (1993) Trends Genet. 9:413-421: Roseanne et al. (1995) Nat. Med. 1:592-594; Albert et al. (1995) Plant J. 7:649-659: Bailey et al. (1992) Plant Mol. Biol. 18:353-361; Odell et al. (1990) Mol Gen. Genet. 223:369-378; and Dale et al. (1991) Proc. Natl. Acad. Sci. USA 88:10558-105620; all of which are herein incorporated by reference); and lox (Albert et al. (1995) Plant J. 7:649-659; Qui et al. (1994) Proc. Natl. Acad. Sci. USA 91:1706-1710; Stuurman et al. (1996) Plant Mol. Biol. 32:901-913; Odell et al. (1990) Mol. Gen. Genet. 223:369-378; Dale et al. (1990) Gene 91:79-85; and Bayley et al. (1992) Plant Mol. Biol. 18:353-361).

For purposes of the present invention, the DNA sequence encoding the recombinase protein and the target sequence for this recombinase protein can be derived from naturally occurring systems (as described above) either by being isolated from the relevant source by use of standard techniques or by being synthesized on the basis of known native sequences. Alternatively, variants or fragments of these DNA sequences may be used as long as they are capable of functioning in the intended manner. The functional variants or fragments may be prepared synthetically and may differ from the wild type sequence in one or more nucleotides.

The mini-Mu plasmid may be constructed such that the internal DNA sequence of the Mu transposable cassette also comprises a nucleotide sequence of interest to be integrated within the host organism's genome to alter the phenotype of the organism. By “nucleotide sequence of interest” is intended a sequence that codes for a desired RNA or protein product, or which itself provides the host cell with a desired property, i.e., a mutant phenotype. Thus, for example, the nucleotide sequence of interest may comprise a sequence encoding a structural or regulatory protein, or may comprise a regulatory sequence such as a promoter. The desired RNA or protein product or regulatory sequence may be heterologous, i.e., foreign, or native to the host cell. If the product or sequence is native to the host cell, transformation of the host cell results in an alteration in phenotype. Where appropriate, the nucleotide sequence of interest may also comprise one or more regulatory elements required for or involved in the expression of the nucleotide sequence encoding the desired RNA or protein product, such as a promoter, a terminator, and the like. The regulatory element(s) may be either heterologous or homologous to the DNA sequence of interest. In this manner, the nucleotide sequence of interest may be constructed as part of an expression cassette as described elsewhere herein and inserted within the internal DNA sequence of the Mu transposable cassette.

Thus, for example, where the host cell is a bacterial or yeast cell, the integration vector of the invention may comprise a nucleotide sequence coding for a polypeptide of interest. Transformation of the bacterial or yeast host cell with such an integration vector allows for targeted insertion of the coding sequence and its regulatory regions within a host genome site that is suitable for maximizing expression of the polypeptide product. Following their selection, transformed host cells are cultivated in a suitable nutrient medium under conditions permitting the expression of the polypeptide, after which the resulting polypeptide is recovered from the culture.

The medium used to culture the cells may be any conventional medium suitable for growing the cells, such as minimal or complex media containing appropriate supplements. Suitable media are available from commercial suppliers or may be prepared according to published recipes (e.g., in catalogues of the American Type Culture Collection). The polypeptide produced by the cells may then be recovered from the culture medium by conventional procedures, including: separating the cells from the medium by centrifugation or filtration; precipitating the proteinaceous components of the supernatant or filtrate by means of a salt, e.g. ammonium sulfate; purification by a variety of chromatographic procedures, e.g. ion exchange chromatography, gel filtration chromatography, affinity chromatography, or the like, depending on the type of polypeptide in question.

Where the host cell is a bacterial or yeast cell to be utilized in production of a polypeptide of interest, preferably the polypeptide is a translocated polypeptide. By “translocated polypeptide” is intended the polypeptide, when expressed, carries a signal sequence which enables it to be translocated across the cell membrane, thereby facilitating its recovery from the culture medium.

Of particular interest are plants that have been transformed with an integration vector of the invention to achieve targeted integration of a nucleotide sequence of interest within the plant's genome. For this intended purpose, the nucleotide sequence of interest may be a regulatory sequence, such as a desirable promoter sequence, or may be a gene coding for a protein whose expression confers a desirable phenotype in the transformed plant. Various changes in phenotype are of interest including modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge. In addition, as our understanding of agronomic traits and characteristics such as yield and heterosis increase, the choice of genes for transformation will change accordingly. General categories of genes of interest include, for example, those genes involved in information, such as zinc fingers, those involved in communication, such as kinases, and those involved in housekeeping, such as heat shock proteins. More specific categories of transgenes, for example, include genes encoding important traits for agronomics, insect resistance, disease resistance, herbicide resistance, sterility, grain characteristics, and commercial products. Genes of interest include, generally, those involved in oil, starch, carbohydrate, or nutrient metabolism as well as those affecting kernel size, sucrose loading, and the like.

Agronomically important traits such as oil, starch, and protein content can be genetically altered in addition to using traditional breeding methods. Modifications include increasing content of oleic acid, saturated and unsaturated oils, increasing levels of lysine and sulfur, providing essential amino acids, and also modification of starch. Hordothionin protein modifications are described in U.S. application Ser. No. 08/838,763, filed Apr. 10, 1997; and U.S. Pat. Nos. 5,703,049, 5,885,801, and 5,885,802, herein incorporated by reference. Another example is lysine and/or sulfur rich seed protein encoded by the soybean 2S albumin described in U.S. Pat. No. 5,850,016, and the chymotrypsin inhibitor from barley, described in Williamson et al. (1987) Eur. J. Biochem. 165:99-106, the disclosures of which are herein incorporated by reference.

Derivatives of the coding sequences can be made by site-directed mutagenesis to increase the level of preselected amino acids in the encoded polypeptide. For example, the gene encoding the barley high lysine polypeptide (BHL) is derived from barley chymotrypsin inhibitor, U.S. application Ser. No. 08/740,682, filed Nov. 1, 1996, and WO 98/20133, the disclosures of which are herein incorporated by reference. Other proteins include methionine-rich plant proteins such as from sunflower seed (Lilley et al. (1989) Proceedings of the World Congress on Vegetable Protein Utilization in Human Foods and Animal Feedstuffs, ed. Applewhite (American Oil Chemists Society, Champaign, Ill.), pp. 497-502; herein incorporated by reference), corn (Pedersen et al. (1986) J. Biol. Chem. 261:6279; Kirihara et al. (1988) Gene 71:359, both of which are herein incorporated by reference), and rice (Musumura et al. (1989) Plant Mol. Biol. 12:123, herein incorporated by reference). Other agronomically important genes encode latex, Floury 2, growth factors, seed storage factors, and transcription factors.

Insect resistance genes may encode resistance to pests that have great yield drag such as rootworm, cutworm, European Corn Borer, and the like. Such genes include, for example, Bacillus thuringiensis toxic protein genes (U.S. Pat. Nos. 5,366,892; 5,747,450; 5,736,514; 5,723,756; 5,593,881; and Geiser et al. (1986) Gene 48:109); lectins (Van Damme et al. (1994) Plant Mol. Biol 24:825); and the like.

Genes encoding disease resistance traits include detoxification genes, such as against fumonosin (U.S. Pat. No. 5,792,931); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylureatype herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene), or other such genes known in the art. The bar gene encodes resistance to the herbicide basta, the nptII gene encodes resistance to the antibiotics kanamycin and geneticin, and the ALS-gene mutants encode resistance to the herbicide chlorsulfuron.

Sterility genes can also be encoded in an expression cassette and provide an alternative to physical detasseling. Examples of genes used in such ways include male tissue-preferred genes and genes with male sterility phenotypes such as QM, described in U.S. Pat. No. 5,583,210. Other genes include kinases and those encoding compounds toxic to either male or female gametophytic development.

The quality of grain is reflected in traits such as levels and types of oils, saturated and unsaturated, quality and quantity of essential amino acids, and levels of cellulose. In corn, modified hordothionin proteins are described in copending U.S. application Ser. No. 08/838,763, filed Apr. 10, 1997, and U.S. Pat. Nos. 5,703,049, 5,885,801, and 5,885,802.

Commercial traits can also be encoded on a gene or genes that could, for example, increase starch for ethanol production or provide expression of proteins. Another important commercial use of transformed plants is the production of polymers and bioplastics such as described in U.S. Pat. No. 5,602,321. Genes such as β-ketothiolase, PHBase (polyhydroxyburyrate synthase), and acetoacetyl-CoA reductase (see Schubert et al. (1988) J Bacteriol. 170:5837-5847) facilitate expression of polyhyroxyalkanoates (PHAs).

Exogenous products include plant enzymes and products as well as those from other sources including prokaryotes and other eukaryotes. Such products include enzymes, cofactors, hormones, and the like. The level of proteins, particularly modified proteins having improved amino acid distribution to improve the nutrient value of the plant, can be increased. This is achieved by the expression of such proteins having enhanced amino acid content.

The scorable marker genes and nucleotide sequences of interest coding for a desired polypeptide product are provided in expression cassettes for expression in the organism of interest. The cassette will include 5′ and 3′ regulatory sequences operably linked to the scorable marker gene and any nucleotide sequence of interest, if appropriate. By “operably linked” is intended a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes. Thus, where both a scorable marker gene and a nucleotide sequence of interest are to be included in the Mu transposable cassette, the sequences can be assembled within the same expression cassette or within different expression cassettes. Such expression cassettes are provided with a plurality of restriction sites for insertion of the nucleotide sequences to be under the transcriptional regulation of the regulatory regions.

The expression cassette comprises in the 5′-3′ direction of transcription, a transcriptional and translational initiation region, a scorable marker gene or nucleotide sequence of interest, and a transcriptional and translational termination region functional in the host organism of interest. The transcriptional initiation region, the promoter, may be native or analogous or foreign or heterologous to the host. Additionally, the promoter may be the natural sequence or alternatively a synthetic sequence. By “foreign” is intended that the transcriptional initiation region is not found in the native organism into which the transcriptional initiation region is introduced. While it may be preferable to express a nucleotide coding sequence using heterologous promoters, the native promoter sequences may be used. Such constructs would change expression levels of the encoded protein in the host organism, thereby altering its phenotype.

A number of promoters can be used in the practice of the invention. The promoters can be selected based on the desired outcome. Thus, the scorable marker gene sequences or nucleotide sequence of interest for coding for a desired polypeptide product can be combined with constitutive, inducible, tissue-preferred, or other promoters for expression in the organism of interest. Such promoters are well known in the art. Any promoter that is functional within the host organism can be used to drive expression of the coding sequence for the scorable marker gene or other nucleotide sequence of interest that comprises a coding sequence for a desired polypeptide product.

For example, where the organism is a plant, useful constitutive promoters include, but are not limited to, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. AppL. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; and 5,608,142.

Inducible promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant PathoL. 89:245-254; Uknes et al. (1992) Plant Cell 4:645-656; and Van Loon (1985) Plant Mol. Virol. 4:111-116. See also the copending application entitled “Inducible Maize Promoters,” U.S. application Ser. No. 09/257,583, filed Feb. 25, 1999, herein incorporated by reference. Other inducible promoters that are expressed locally at or near the site of pathogen infection, including, for example, those described in Marineau et al. (1987) Plant Mol. Biol. 9:335-342; Matton et al. (1989) Molecular PlantMicrobe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant Path. 41:189-200).

Chemical-regulated promoters can be used to modulate the expression of a gene through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to: the maize In 2-2 promoter, which is activated by benzenesulfonamide herbicide safeners; the maize GST promoter, which is activated by hydrophobic electrophilic compounds that are used as pre-emergent herbicides; and the tobacco PR-1 a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced expression of a coding sequence within a particular tissue. Tissue-preferred promoters operable in plants, for example, include those described in Yamamoto et al. (1997) Plant J. 12(2)255-265; Kawamata et al. (1997) Plant Cell Physiol. 38(7):792-803; Hansen et al. (1997) Mol. Gen Genet. 254(3):337-343; Russell et al. (1997) Transgenic Res. 6(2):157-168; Rinehart et al. (1996) Plant Physiol 112(3):1331-1341; Van Camp et al. (1996) Plant Physiol 112(2):525-535; Canevascini et al. (1996) Plant Physiol. 112(2):513-524; Yamamoto et al. (1994) Plant Cell Physiol. 35(5):773-778; Lam (1994) Results Probl. Cell differ. 20:181-196; Orozco et al. (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al (1991) Mol. Gen. Genet. 262:141-144; Proudfoot (1991) Cell 64:671-674; Sanfacon et al (1991) Genes Dev. 5:141-149; Mogen et al. (1990) Plant Cell 2:1261-1272; Munroe et al. (1990) Gene 91:151-158; Ballas et al. (1989) Nucleic Acids Res. 1 7:7891-7903; and Joshi et al. (1987) Nucleic Acid Res. 15:9627-9639.

Where the transformed organism is a plant, the gene(s) may be optimized for increased expression. That is, the genes can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary MRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. Sci. USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, N.Y.), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Thus the integration vectors of the invention may be engineered to comprise a nucleotide sequence of interest for subsequent targeted integration into the genome of a host organism. In this manner, the present invention provides a method for creating or enhancing the expression of a nucleotide sequence of interest in a host organism. Transformation of an organism with an integration vector of the invention which comprises the nucleotide sequence of interest within its Mu transposable cassette results in targeted insertion of the nucleotide sequence of interest within a predetermined site in the host organism's genome.

Alternatively, the integration vectors of the invention are useful for inactivating or knocking out a gene within a host organism, which may be a naturally occurring gene or one that has previously been transformed and integrated within the host organism's genome. Thus the invention provides a method for producing knockout mutants or a knockout mutation within a host organism. In this manner, for example, the non-Mu plasmid DNA domain of a mini-Mu plasmid can comprise a region of DNA that shares homology with a predetermined binding sequence that is adjacent to the gene targeted for inactivation. Transformation of a host cell with the integration vector derived from this mini-Mu plasmid results in insertion of the Mu transposable cassette into the predetermined target site, causing the gene to be inactivated.

Previously integrated transgenes of particular interest for inactivation include, but are not limited to, herbicide, scorable marker genes, and the like. Other genes of interest for targeted inactivation include, but are not limited to, genes having mutations that are deleterious to the host organism.

The gene-targeting methods of the invention can be utilized to genetically modify any organism of choice. By organism of choice is intended prokaryotic organisms, such as Escherichia coli, Bacillus subtilis, Pseudomonas species, etc., or eukaryotic organisms, including yeast, fungi, mammal, and more particularly plant species.

The present invention may be used for transformation of any plant species, including, but not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp.), and members of the genus Cucumis such as cucumber (C. sativus), cantaloupe (C. cantalupensis), and musk melon (C. melo). Ornamentals include azalea (Rhododendron spp.), hydrangea (Macrophylla hydrangea), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (Pinus elliotii), ponderosa pine (Pinus ponderosa), lodgepole pine (Pinus contorta), and Monterey pine (Pinus radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Preferably, plants of the present invention are crop plants (for example, corn, alfalfa, sunflower, Brassica, soybean, cotton, safflower, peanut, sorghum, wheat, millet, tobacco, etc.), more preferably corn and soybean plants, yet more preferably corn plants.

Methods for introducing constructs comprising DNA into prokaryotic or eukaryotic host cells are well known in the art. Transformation of a cell with DNA requires that the DNA construct be physically placed within the host cell. Current transformation procedures utilize a variety of techniques to introduce DNA into a cell. These include, but are not limited to, calcium phosphate transfection, DEAE-dextran-mediated transfection, cationic lipid-mediated transfection, electroporation, transduction, infection, lipofection, protoplast fusion, microparticle bombardment, and other techniques such as those found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989). In one form of transformation, the DNA is microinjected directly into cells though the use of micropipettes. Alternatively, high velocity ballistics or microprojectile bombardment can be used to introduce DNA and associated molecules and proteins (e.g., spermidine) into the cell. In another form, the cell is permeabilized by the presence of polyethylene glycol, thus allowing DNA and other molecules to enter the cell through diffusion. DNA can also be introduced into a cell by fusing protoplasts with other entities which contain DNA. These entities include minicells, cells, lysosomes or other fusible lipid-surfaced bodies. Electroporation is also an accepted method for introducing DNA solutions into a cell. In this technique, cells are subject to electrical impulses of high field strength which reversibly permeabilizes biomembranes, allowing the entry of exogenous DNA solutions. Any such method for directly introducing DNA and/or protein into a prokaryotic or eukaryotic cell can be used to introduce the novel integration vectors of the present invention to obtain transformed cells comprising the Mu transposable element integrated within a predetermined target site in the host organism's genome. In many transformation techniques, those of skill in the art are aware that more than one type of vector or nucleic acid preparation may be included in the transformation mixture and thereby be introduced into the host cell. The transformation mixture may also include other ingredients to enhance the transformation process, such as proteins, surfactants, etc. For example, other selectable marker genes may be cotransformed with the integration vector of interest to aid selection or identification of transformed cells.

Thus, the disclosed integration vectors of the present invention may be introduced into the nucleus of a plant cell by any method available in the art. In this manner, genetically modified plants, plant cells, plant tissue, seed, and the like can be obtained. These constructs may be introduced into the plant by one or more techniques typically used for direct DNA delivery into cells. Such protocols may vary depending on the type of plant or plant cell targeted for gene modification. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; Tomes et al. (1995) “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al. (1988) Biotechnology 6:923-926). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In vitro Cell Dev. Biol. 27P:175-182 (soybean); Singh et al. (1998) Theor. Appl. Genet. 96:319-324 (soybean); Datta et al (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, N.Y.), pp. 197-209 (pollen); Kaeppler et al. (1990) Plant Cell Reports 9:415-418 and Kaeppler et al. (1992) Theor. Appl. Genet. 84:560-566 (whisker-mediated transformation); D'Halluin et al. (1992) Plant Cell 4:1495-1505 (electroporation); Li et al. (1993) Plant Cell Reports 12:250-255 and Christou and Ford (1995) Annals of Botany 75:407-413 (rice); all of which are herein incorporated by reference. In one embodiment of the invention, the integration vector is transformed into a host plant or plant cell using microinjection.

The plant cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure that expression of the desired phenotypic characteristic has been achieved.

The following examples are offered by way of illustration and not by way of limitation.

### EXPERIMENTAL

### Example 1

### Construction of RecA-Coated Single-Stranded Cleaved Donor Complex for Use in Producing a Knockout Mutant

An integration vector comprising a cleaved donor complex and an attached RecA-coated, single-stranded navigator element (referred to herein as “ssCDC”) is constructed from a mini-Mu plasmid using the standard in vitro reactions. The mini-Mu plasmid has the reporter gene GFP operably linked to the ubiquitin promoter, and the selectable marker PAT inserted within the Mu transposable cassette. This selectable marker confers resistance to the herbicide Bialaphos (Wohlleben et al. (1988) Gene 70:25-37). The non-Mu plasmid domain of the mini-Mu plasmid is constructed such that it has a region of nucleotides that shares homology with a gene targeted for disruption within the genome of a plant of interest. In this example, the gene is a GUS transgene whose site of integration within a maize plant's genome has been previously determined. Thus the region of nucleotides within the non-Mu plasmid DNA shares homology with the maize genomic sequence of the previously integrated GUS gene.

In the presence of Mg²⁺, MuA binds to the binding sites within the ends of the Mu transposable cassette, forming an active CDC having a MuA tetrameric core. Following formation of this active CDC, the CDC is digested by a restriction enzyme, such as EcoRI, to linearize the non-Mu plasmid DNA domain of the CDC. This generates two flanking double-stranded non-Mu plasmid DNA sequences up to the MuA tetramer. Further treatment of the non-Mu plasmid DNA sequences with the exonuclease ExoIII which digests DNA in the 3′ to 5′ direction converts at least one of these flanking double-stranded non-Mu sequences into a single-stranded non-Mu plasmid DNA sequence, which may extend up to the MuA tetramer.

The single-stranded CDC is then incubated with the E. coli RecA protein using methods known in the art to obtain the integration vector, a RecA-coated ssCDC. See, for example, Sena and Zarling (1993), Nature Genetics 3: 365-372, and U.S. Pat. Nos. 5,223,414, and 5,273,881. This integration vector can then be used to disrupt GUS expression by transformation into maize carrying the appropriate GUS transgene.

### Example 2

### Construction of a RecA-Coated Single-Stranded Cleaved Donor Complex for Use in Targeted Integration of a Nucleotide Sequence of Interest into a Maize Plant's Genome

An integration vector comprising a cleaved donor complex and an attached RecA-coated, single-stranded navigator element (referred to herein as “ssCDC”) is generated as in Example 1. However, the mini-Mu plasmid in this instance is constructed with a nucleotide sequence coding for a heterologous protein operably linked to the ubiquitin promoter inserted within the Mu transposable cassette. The Mu transposable cassette also has the PAT selectable marker operably linked to the ubiquitin promoter. The non-Mu plasmid DNA domain of the mini-Mu plasmid contains a region of nucleotides that share homology with a predetermined site adjacent to a predetermined target site within a maize plant'genome. The predetermined target site has been previously identified as a preferred site of integration of the foreign DNA on the basis of its minimal effect on endogenous gene expression and minimal position effects.

Following formation of an active CDC, a RecA-coated single-stranded CDC is obtained as outlined in Example 1. This CDC is then transformed into maize and transformed plants are identified by an appropriate screening or selection technique. The transformed plants are then tested for the presence of the heterologous protein.

### Example 3

### In vitro Gene-Targeting Assay

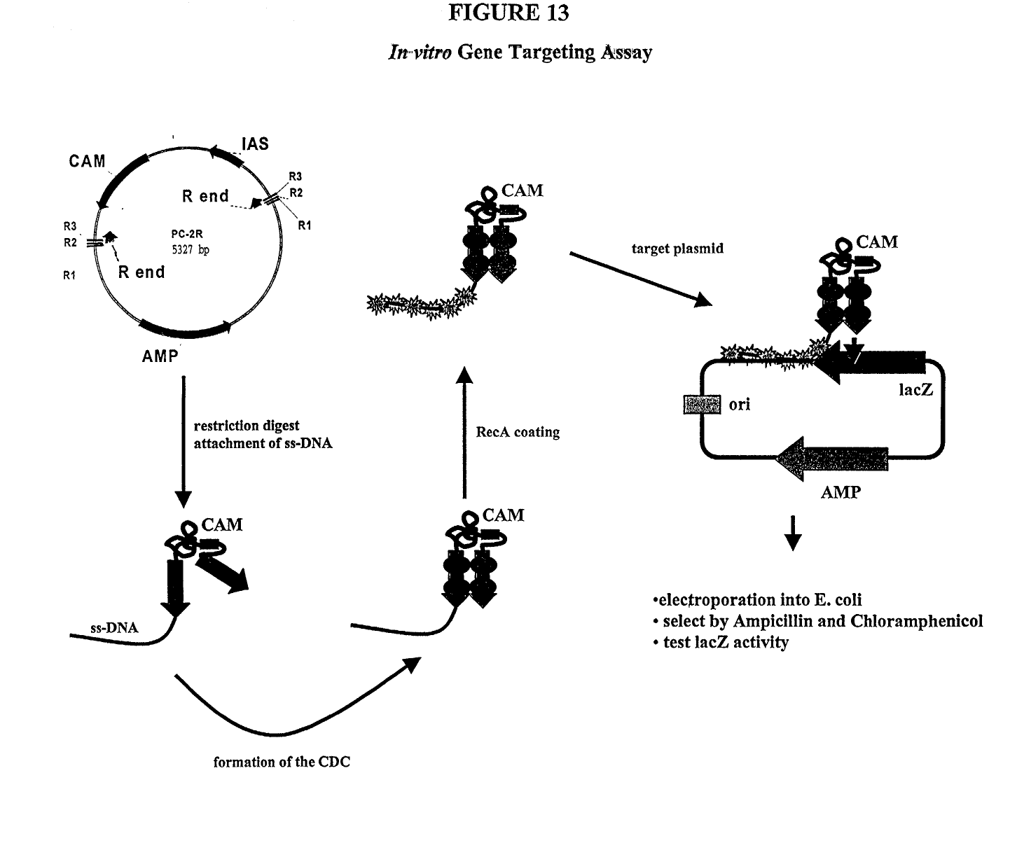

A derivative mini-Mu plasmid such as PC-2R (diagrammed in FIG. 13) is constructed with a Mu transposable cassette that comprises two right end recognition sequences flanking the Mu internal activation sequence (IAS) and a chloramphenicol resistance gene (“CAM”; see FIG. 13). This plasmid is subjected to restriction digestion to generate a precleaved mini-Mu plasmid that has 5′ overhang sequences that immediately flank the extreme ends of the Mu right end recognition sequences. A single-stranded DNA (ss-DNA) navigator element comprising a region of nucleotides having homology with a sequence adjacent to the lacZ gene is then ligated into one of these overhang sequences. The resulting construct is incubated with MuA transposase in vitro to obtain a CDC attached to a single-stranded DNA navigator element. Following RecA coating of the single-stranded DNA navigator element, the resulting integration vector is electroporated into E. coli together with a lacZ-containing plasmid such as pUC19. Transfection events are selected with ampicillin and chloramphenicol, and altered lacZ activity is tested.

### Example 4

### Optimization of Conditions for In Vitro Targeting Experiments

In vitro targeting experiments were designed to demonstrate that modified CDC complexes with a navigator molecule attached are competent in transpositions. Generally, a mixture of CDC and target plasmid DNA was used. The target plasmid DNA was pBIN19, a publicly available plasmid which contains the lacZ gene. When CDC complex integrates into the lacZ gene, the lacZ gene is disrupted and white instead of blue bacterial colonies are formed.

The navigator molecule used in these experiments was an oligonucleotide with 60 bp of sequence complementary to the lacZ gene on the pBIN19 plasmid. A biotin molecule was attached to the 5′ end of this oligonucleotide, which was then incubated with avidin and coated with RecA, a bacterial protein known to stimulate recombination between homologous DNA sequences. The PC-4 SpeI/BglII fragment (as described in Example 5 below) was biotinylated and incubated with MuA protein to form the active CDC complexes. These active CDC complexes were then incubated in a solution containing the navigator elements in order to attach the navigator elements to the CDC complexes. Three different incubation conditions were used, as follows:

Condition 1-0.5 μg pBIN

Condition 2-0.1 μg pBIN plus 0.4 μg HindIII digested lambda DNA (carrier DNA)

Condition 3-0.01 μg pBIN plus 0.5 μg HindIII digested lambda DNA (carrier DNA)

Results were as follows:

TABLE 1
Efficiency of Targeting
(# of white colonies)/total colonies

| Procedure | Condition 1 | Condition 2 | Condition 3 |
|---|---|---|---|
| Total number of colonies | (0)/1 | (2)/70 | (5)/45 |
| Percentage white colonies | N/A | 2.9% | 11.1% |

Thus, the ratio of white to blue colonies increased up to 11% under Condition 3; this indicates a preferential integration of the CDC complex into the lacZ gene under certain conditions.

### Example 5

### Demonstration of Transpositional Functionality of CDC Complexes In Vivo

Experiments conducted in vivo in E. coli demonstrated that attachment of an ss-DNA navigator to the pre-cleaved donor complex does not affect transposition activity of the complex.

Two modifications to the CDC complex were evaluated: 1) attachment of a single-stranded DNA navigator to the CDC complexes through annealing of complementary sequences; and 2) attachment of a single-stranded DNA navigator to the CDC complexes through a biotin/streptavidin bridge. The CDC complexes were either formed before electroporation into E. coli, or they were formed inside E. coli cells expressing an inducible MuA protein.

Standard DH5α E.coli strain was used to introduce the pre-formed CDC complexes. A strain containing a genome-integrated lacZ gene was electroporated with an appropriate plasmid to produce MuA-expressing bacterial cells. This plasmid contained an origin of replication, neomycin phosphotransferase gene, and the MuA coding sequence controlled by the T7 promoter.

Cutting the PC-4 vector with SpeI and BglII generated a DNA fragment that contained the chloramphenicol acetyltransferase gene (“CAM” or “CAM-R,” which confers chloramphenicol resistance) embedded into Mu bacteriophage sequences and flanked by two MuA binding sites.

A single-stranded DNA navigator was designed to contain 100 bp homology to the lacZ coding region on pUC19. Ten nucleotides at the 3′ end of this oligonucleotide were complementary to the transient DNA adaptor used for connecting the navigator to the SpeI restriction site of the CDC. The transient adaptor (0.3 micrograms (μg)) and 2.2 ug lacZ ss-DNA navigator (lacZ-100) were annealed in the annealing buffer (10 mM Tris, pH 7.5, 50 mM NaCl, 0.1 mM EDTA) in a total volume of 100 microliters (μl). One μl of the annealing reaction was mixed with 1 μg of the pre-cleaved CDC and ligated overnight at 16° C. (3 μls of T4 ligase and 5 μls 10×ligation buffer in a total volume of 50 μls). The ligase was heat-inactivated for 10 min at 75° C. and DNA complexes precipitated in ethanol. Subsequently, the ligation products were restricted with BglII and purified using the QIAGEN PCR purification kit according to the manufacturer's instructions. Formation of the CDC/ ss-DNA navigator complexes was verified by observation of a shift in the DNA band position on agarose gels. The CDC complex with a navigator was extracted from an agarose gel as a shifted band compared to the original position of CDC.

For delivery of the pre-formed CDC complexes containing the MuA monomers attached to the binding sites, 20 nanograms (ng) of the PC-4 SpeI/BglII fragment (with or without a navigator) was incubated with 0.22 μg MuA protein in a buffer solution containing 25 mM HEPES (pH 7.6), 130 mM NaCl, 10 mM MgC12, 15% DMSO, and 15% glycerol. The reaction (total volume 20 μs) was incubated at 30° C. for 1 hr, followed by DNA precipitation in ethanol and re-dissolving in 10 μls of water.

Two μls of the CDC solution was used to electroporate E. coli cells under standard conditions. Bacterial cells were grown on 2xYT medium containing 100 μg/ml carbenicillin, 30 μg/ml chloramphenicol, IPTG, and X-Gal. The number of blue and white colonies was estimated after overnight incubation at 37° C. Blue colonies indicate that the lacZ gene previously integrated into the E. coli genome remains active; white colonies indicate that the previously integrated lacZ gene has been inactivated. Results obtained were:

| | Number of colonies | | |
|---|---|---|---|
| Treatment | Blue | White | Total |
| PC-4 fragment only | 0 | 0 | 0 |
| pUC19 DNA only | 0 | 0 | 0 |
| CDC complex | 16 | 4 | 20 |
| CDC complex + navigator | 26 | 5 | 31 |

Thus, attachment of a ssDNA navigator to the precleaved CDC did not affect transposition activity of the complex.

Further experiments showed that the functional CDC complex can be formed inside E.coli expressing MuA.

For introduction of the CDC complexes, an E.coli strain containing a MuA-expressing plasmid was made electroporation-competent under MuA inducible and noninducible conditions. A high level of MuA expression was achieved by growing bacteria in the presence of 0.5 mM IPTG. One hundred ng of the PC-4 SpeI/BglII fragment was electroporated into such cells under standard electroporation conditions. A positive control treatment included 20 ng of the PC-4 SpeI/BglII fragment incubated with 0.2 ng of MuA protein before electroporation. Two-hundred ng of the PC-4 fragment and 4 ng of the lacZ-100/biotin/streptavidin navigator were incubated in 2.5 mM Tris-HCl buffer (pH 7.5) and 5 mM NaCl for 1 hr at room temperature to form a biotin/strepavidin bridge between the navigator and CDC . The same conditions were used to produce a stock solution of the lacZ-100/biotin/strepavidin complex by incubating 18 μg of ss-DNAbiotin and 27 μg of streptavidin in a total volume of 40 μls. Transformed bacterial cells were grown overnight on 2xYT medium supplemented with 50 μg/ml kanamycin and 10 μg/ml chloramphenicol. Results showed that production of MuA in the host cell dramatically increased the number of kanamycin-resistant and chloramphenicol-resistant colonies:

Number of colonies

| Treatment | Uninduced MuA | Induced MuA |
|---|---|---|
| BL21(DE3) cells only | 0 | 0 |
| CDC without navigator - 1 | 1 | 240 |
| CDC without navigator - 2 | 3 | 204 |
| CDC with navigator | 0 | 58 |

Thus, results showed that a functional CDC complex could be formed in vivo in a host organism where MuA was present.

### Example 6

### Transformation by Microprojectile Bombardment and Regeneration of Transgenic Maize

Immature maize embryos from greenhouse donor plants are bombarded with an integration vector obtained in Example 1 or 2. Where the active CDC is a stripped-down version, embryos are co-bombarded with plasmid comprising a ubi::NLS::MuA::pinII construct (ubiquitin promoter driving expression of a region encoding nuclear localization signal operably linked to MuA protein). Transformation is performed as follows. Media recipes follow below.

Preparation of Target Tissue

The ears are surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

Preparation of DNA

The integration vector obtained in Example 1 or 2 is precipitated onto 1.1 μm (average diameter) tungsten pellets using a standard CaCl₂ precipitation procedure optimized for delivery of the DNA-protein complex. The standard procedure (prior to optimization) is as follows:

100 μl prepared tungsten particles in water

10 μl (1 μg) DNA in TrisEDTA buffer (1 μg total)

100 μl 2.5 M CaC1₂

10 μl 0.1 M spermidine

In the case of the present experiment, the integration vector comprises not only DNA but also protein, which together form a DNA-protein complex.

In the standard procedure, each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA-protein particles are briefly sonicated and 10 μl spotted onto the center of each macrocarrier and allowed to dry about 2 minutes before bombardment.

Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Where the integration vector is obtained from Example 1, plants are monitored and scored for disruption of GUS expression. Where the integration vector is obtained from Example 2, plants are monitored and scored for activity of the heterologous protein.

Bombardment and Culture Media

Bombardment medium (560Y) comprises 4.0 g/l N6 basal salts (SIGMA C-1416), 1.0 ml/l Eriksson's Vitamin Mix (1000X SIGMA-1511), 0.5 mg/l thiamine HCl, 120.0 g/l sucrose, 1.0 mg/l 2,4-D, and 2.88 g/l L-proline (brought to volume with D-I H₂O following adjustment to pH 5.8 with KOH); 2.0 g/l Gelrite (added after bringing to volume with D-I H₂O); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000X SIGMA-1511), 0.5 mg/l thiamine HCl, 30.0 g/l sucrose, and 2.0 mg/l 2,4-D (brought to volume with D-I H₂O following adjustment to pH 5.8 with KOH); 3.0 g/l Gelrite (added after bringing to volume with D-I H₂O); and 0.85 mg/l silver nitrate and 3.0 mg/l bialaphos(both added after sterilizing the medium and cooling to room temperature). Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-I H₂O) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-I H₂O after adjusting to pH 5.6); 3.0 g/l Gelrite (added after bringing to volume with D-I H₂O); and 1.0 mg/l indoleacetic acid and 3.0 mg/l bialaphos (added after sterilizing the medium and cooling to 60° C). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-I H₂O), 0.1 g/l myo-inositol, and 40.0 g/l sucrose (brought to volume with polished D-I H₂O after adjusting pH to 5.6); and 6 g/l bacto-agar (added after bringing to volume with polished D-I H₂O), sterilized and cooled to 60° C.

### Example 7

### Soybean Embryo Transformation

Soybean embryos are bombarded with an active CDC attached to a navigator element as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can be maintained in 35 ml liquid media on a rotary shaker, 150 rpm, at 26° C. with fluorescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327:70-73, U.S. Pat. No. 4,945,050). A Du Pont Biolistic PDS1000/HE instrument (helium retrofit) can be used for these transformations.

A selectable marker gene that can be used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. An integration vector, said vector being a navigator-attached cleaved donor complex (CDC), wherein said navigator-attached CDC comprises a Mu transposable cassette from a precleaved mini-Mu plasmid and at least one navigator element attached to a non-Mu plasmid DNA sequence flanking said Mu transposable cassette, wherein said navigator element comprises a region of nucleotides that is complementary to a predetermined binding sequence within the genome of an organism of interest, said predetermined binding sequence being adjacent to a predetermined target site within said genome.

2. The integration vector of claim 1, wherein said navigator element is composed of a synthetic analog of a nucleic acid.

3. The integration vector of claim 1, wherein said navigator element is composed of peptide nucleic acid.

4. The integration vector of claim 1, wherein said navigator element is composed of protein.

5. The integration vector of claim 1, wherein said navigator element is composed of DNA.

6. The integration vector of claim 1, wherein said navigator element is composed of a combination of two or more of the group consisting of nucleic acid, synthetic analog of nucleic acid, peptide nucleic acid, protein, RNA, or DNA.

7. An integration vector , said vector comprising a cleav ed donor complex (CDC) and at least one navigator element attached to said CDC, wherein said CDC comprises a Mu transposable cassette from a mini-Mu plasmid and wherein said navigator element comprises at least one single-stranded non-Mu plasmid DNA sequence coated with a RecA-like protein, wherein at least one of said single-stranded non-Mu plasmid DNA sequences comprises a region of nucleotides that is complementary to a predetermined binding sequence within the genome of an organism of interest, said predetermined binding sequence being adjacent to a predetermined target site within said genome.

8. A host cell stably transformed with the integration vector of claim 1.

9. The host cell of claim 8, wherein said host cell is a plant cell.

10. The host cell of claim 9, wherein said plant cell is from a monocot.

11. The host cell of claim 9, wherein said plant cell is from a dicot.

12. A host organism stably transformed with the integration vector of claim 1, said active CDC containing a Mu transposable cassette.

13. The host organism of claim 12, wherein said host organism is a plant.

14. The host organism of claim 13, wherein said plant is a monocot.

15. The host organism of claim 13, wherein said plant is a dicot.

16. A method for stably integrating a nucleotide sequence of interest into a target site within the genome of an organism, said method comprising transforming said organism with a navigator-attached cleaved donor complex (CDC), wherein said navigator-attached CDC comprises a Mu transposable cassette from a precleaved mini-Mu plasmid and at least one navigator element attached to a non-Mu plasmid DNA sequence flanking said Mu transposable cassette, wherein said Mu transposable cassette comprises said nucleotide sequence of interest, and wherein said navigator element comprises a region of nucleotides that is complementary to a predetermined binding sequence within the genome of an organism of interest, said predetermined binding sequence being adjacent to said target site, wherein said navigator-attached CDC hybridizes to said predetermined binding sequence adjacent to said target site, whereby said Mu transposable cassette comprising said nucleotide sequence of interest is inserted within said target site.

17. A method for producing a mutation in a target gene within the genome of an organism, said method comprising transforming said organism with a navigator-attached cleaved donor complex (CDC), wherein said navigator-attached CDC comprises a Mu transposable cassette from a precleaved mini-Mu plasmid and at least one navigator element attached to a non-Mu plasmid DNA sequence flanking said Mu transposable cassette, wherein said navigator element or navigator elements comprise a region of nucleotides that is complementary to a predetermined binding sequence within the genome of an organism of interest, said predetermined binding sequence being adjacent to said target gene, wherein said navigator-attached CDC hybridizes to said predetermined binding sequence adjacent to said target gene, whereby said Mu transposable cassette is inserted within said target gene thereby producing said mutation in said target gene.

18. The method of claim 17, wherein said organism is a plant.

19. The method of claim 17, wherein said mutation is a partial or complete deletion of the regulatory region and coding region of said target gene, whereby expression of said gene is disrupted.
